(19) United States
(12) Patent Application Publication
(10) Pub. No.: US 2020/0093158 A1
(43) Pub. Date: Mar. 26, 2020

(54) COMPOSITIONS AND COMBINATIONS FOR USE AS FOOD SUPPLEMENTS FOR ANIMALS

(71) Applicant: OmniGen Research, LLC, Corvallis, OR (US)

(72) Inventors: David Calabotta, Quincy, IL (US); James D. Chapman, Macon, GA (US); Tim Costigan, Quincy, IL (US); Neil E. Forsberg, Corvallis, OR (US); Wendell Knehans, St. Louis, MO (US); Derek McLean, Corvallis, OR (US); Steven B. Puntenney, Corvallis, OR (US); Angela D. Rowson, Quincy, IL (US)

(73) Assignee: OmniGen Research, LLC, Corvallis, OR (US)

(21) Appl. No.: 16/532,877

(22) Filed: Aug. 6, 2019

Related U.S. Application Data
(63) Continuation of application No. 15/475,489, filed on Mar. 31, 2017, now abandoned.
Application No. 15/475,489 is a continuation-in-part of application No. PCT/US2015/053439, filed on Oct. 1, 2015.
(60) Provisional application No. 62/058,461, filed on Oct. 1, 2014.

(51) Int. Cl. A23K 50/50; A23K 20/163; A23K 50/60; A23K 40/25; A23K 10/30; A23K 20/10; A23K 20/20; A23K 20/28; A23K 50/20; A23K 50/10; A23K 50/40; A23K 50/80; A23K 20/174; A61K 31/715; A61K 31/716; A61K 33/00; A61K 33/24; A61K 36/185; A61K 36/88; A61K 38/47; A61K 45/06

(52) U.S. Cl. CPC A23K 50/50; Y02A 40/818; A23K 50/60; A23K 40/25; A23K 10/30; A23K 20/10; A23K 20/20; A23K 20/28; A23K 50/20; A23K 50/10; A23K 50/40; A23K 50/80; A23K 20/174; A61K 31/715; A61K 31/716; A61K 33/00; A61K 33/24; A61K 36/185; A61K 36/88; A61K 38/47; A61K 45/06; C12Y 302/01006; Y02P 60/877; A23K 20/163

(57) ABSTRACT

Disclosed herein are embodiments of compositions and combinations that can be used in combination with animal food. In some embodiments, the combinations comprise various compositions that can provide health benefits for animals, such as domestic or companion animals and/or feed animals. The combinations can be used to help maintain and/or promote animal health and well-being, and in some embodiments can help promote an increase in animal longevity.

### CROSS REFERENCE TO RELATED APPLICATION

This is a U.S. continuation of U.S. patent application Ser. No. 15/475,489, filed on Mar. 31, 2017, which is a continuation-in-part of International Application No. PCT/US2015/053439, filed on Oct. 1, 2015, which was published in English under PCT Article 21(2), which in turn claims the benefit of U.S. Provisional Application No. 62/058,461, filed Oct. 1, 2014, all of which are incorporated herein by reference in their entirety.

### FIELD

The present disclosure concerns compositions and combinations of such compositions for use as supplements alone or in combination with a feedstuff or companion animal food. Also disclosed herein are methods of using the combinations to help increase animal longevity and help support a healthy animal immune system.

### SUMMARY

Disclosed herein are embodiments of a combination, comprising a primary composition comprising mineral clay and silica, β-glucan, or a combination thereof; and any one or more of yucca, quillaja, a direct-fed microbial, a vitamin D species, a chromium compound, or a plant extract. In some embodiments, the primary composition can further comprise mannans. In additional embodiments, the primary composition can further comprise an endoglucanohydrolase. Exemplary primary compositions can comprise 1-40 wt % silica, 0.5-25 wt % glucan and mannans, such as 1-25 wt % glucan and mannans, and 30-92 wt % mineral clay. In some embodiments, the combination can comprise each of yucca, quillaja, a direct-fed microbial, a vitamin D species, and a plant extract, and may further comprise a chromium compound. In additional embodiments, the combination can comprise Quillaja saponaria, Yucca schidigera, B. coagulans, 25-hydroxy vitamin D3, a polyphenol, a chromium compound, or any combination thereof, and in other embodiments, the combination can comprise Quillaja saponaria, Yucca schidigera, B. coagulans, and 25-hydroxy vitamin D3. Combination embodiments disclosed herein can comprise the primary composition and any one or more of the yucca, quillaja, direct-fed microbial, vitamin D species, a chromium compound, or plant extract in a ratio from 1,000:1 to 1:1,000 by weight. In exemplary embodiments, a combination for promoting an increase in an animal's lifespan or promoting a healthy immune system is disclosed and can comprise a primary composition comprising 35-92% mineral clay and 1-40% silica, 0.5-30% β-glucan, such as 1-30% β-glucan, or a combination thereof, and any one or more of yucca, quillaja, a direct-fed microbial, a vitamin D species, a chromium compound, or a plant extract in an amount ranging from 5 ppm to 2,000 ppm based on the total dry weight basis of the combination. This combination causes an increase or decrease in a level of an immune system biomarker or an inflammation biomarker in the animal. In some embodiments, the increase or decrease ranges from at least 5% to 600%.

Some embodiments can concern combinations comprising a metal carbonate, kelp, a niacin supplement, a vitamin B-12 supplement, biotin, d-calcium pantothenate, choline chloride, thiamine mononitrate, pyridoxine hydrochloride, menadione dimethylpyrimidinol bisulfite, riboflavin-5-phosphate, folic acid, soybean oil, calcium aluminosilicate, rice hulls, mineral oil, or any combination thereof. In some embodiments, the primary composition can be formulated as a powder, a granule, a pellet, a solution, or a suspension. In some embodiments, the combination is formulated as a powder, a granule, a pellet, a solution, a suspension, or a combination thereof.

Particular disclosed embodiments of the combination can further comprise a feedstuff or companion animal food. Exemplary combinations can comprise a primary composition, comprising a β-glucan, silica, mineral clay, and mannans; any one or more of Quillaja saponaria, Yucca schidigera, B. coagulans, 25-hydroxy vitamin D3, chromium compound, or a combination thereof; and a feedstuff or companion animal food.

Also disclosed herein are methods of administering to an animal (a) a primary composition comprising mineral clay and silica, β-glucan, or a combination thereof; and (b) any one or more of yucca, quillaja, a direct-fed microbial, a vitamin D species, a chromium compound, or a plant extract.

While the combination can be administered to animals of all ages, certain other method embodiments concern selecting a young or geriatric animal, and administering to the animal a combination comprising a primary composition comprising mineral clay and silica, β-glucans, or a combination thereof, and any one or more of yucca, quillaja, a direct-fed microbial, a vitamin D species, a chromium compound, or a plant extract; wherein administering the combination to the animal helps promotes an increase in the animal's lifespan relative to an animal that is not administered the composition. Such embodiments can comprise using a primary composition that further comprises mannans, an endoglucanohydrolase, or both. In some embodiments, the method can further comprise administering to the animal a companion animal feed or feedstuff. The feedstuff can be a feed ration, a mineral supplement, a protein supplement, a premix, molasses, a liquid feed, water, or any combination thereof. In some embodiments, the feedstuff or companion animal food can be admixed with the combination prior to administration. In some embodiments, the animal can be a companion animal, such as a canine or feline. In other embodiments, the animal can be a feed animal, such as a domestic fowl or a cow. In yet other embodiments, the animal can be a pig, a fish, a reptile, a crustacean, a rabbit, a sheep, a goat, a deer, a bison, a buffalo, an alpaca, a horse, a donkey, or a llama.

In some embodiments, administering the combination to the animal ameliorates at least one deleterious symptom or sign observed or measured in the animal. In another embodiment, administering the combination to the animal delays onset of the at least one deleterious symptom or sign observed or measured in the animal. Exemplary deleterious symptoms or signs can include any symptom or sign associated with dermatitis, atopy, flea-allergy dermatitis, food allergy dermatitis, contact dermatitis, juvenile dermatitis, osteoarthritis, conjunctivitis/uveitis, pyometra, prostatitis, orchitis cystitis, renal disease, Pemphigus vulgaris/foliaceus, lupus, autoimmune hemolytic anemia, rheumatoid arthritis, Addison's Disease, Cushing's disease, hyper and hypothyroidism, diabetes mellitus, diabetes insipidus, inflammatory bowel disease, hemorrhagic gastroenteritis, otitis, distemper virus, parvovirus, coronavirus, herpesvirus, influenza virus, hepatitis virus, Salmonellosis, Bordetella bronchiseptica, Lyme's disease, leptospirosis, brucellosis, blastomycosis, aspergillosis, Cryptococcus, coccidioidomycosis, microsporum/trichophyton, histoplasmosis, coccidiosis, giardiasis, hookworms, roundworms, whipworms, tapeworms, mange, meningitis-arteritis/encelphalitis, acquired myasthenia gravis, lymphosarcoma, osteosarcoma, hemangiosarcoma, mast cell tumor, squamous cell carcinoma, melanoma, leukemia, mammary tumors, lung cancer, testicular cancer, transitional cell carcinoma, brain tumor, laminitis, founder, heart disease, epilepsy, hepatitis, pancreatitis, gingivitis, impaired cognitive function and/or memory, insulin resistance in the brain, and combinations thereof. In some embodiments, the at least one deleterious symptom or sign comprises an aberrant immune system biomarker or an aberrant inflammation biomarker.

The foregoing and other objects and features of the disclosure will become more apparent from the following detailed description, which proceeds with reference to the accompanying figures.

### BRIEF DESCRIPTION OF THE DRAWINGS

FIG. 1 is a bar graph showing the effects of a composition embodiment, Composition A (CTP-5), a plant extract, grape pomace extract (GPE), and the combination of the two (CTP-5+GPE) on L-selectin mRNA concentration in rat neutrophils following a 6-day study. L-selectin mRNA was expressed as a proportion of a housekeeping gene (β-actin mRNA).

FIG. 2 is a bar graph showing the effects a composition embodiment, Composition A (CTP-5), a plant extract, grape pomace extract (GPE), and the combination of the two (CTP-5+GPE) on IL8R mRNA concentration in rat neutrophils following a 6-day study. IL-8R mRNA was expressed as a proportion of a housekeeping gene (β-actin mRNA).

FIG. 3 is a bar graph showing the effects of a composition embodiment, Composition A (CTP-5), a plant extract, grape pomace extract (GPE), and the combination of the two (CTP-5+GPE) on L-selectin mRNA concentration in rat neutrophils following a 28-day study. The y-axis represents L-selectin mRNA/beta actin mRNA (with control set to 1.0). L-selectin mRNA was expressed as a proportion of a housekeeping gene (β-actin mRNA).

FIG. 4 is a bar graph showing the effects of a composition embodiment, Composition A (CTP-5), a plant extract, grape pomace extract (GPE), and the combination of the two (CTP-5+GPE) on IL8R mRNA concentration in rat neutrophils following a 28-day study. The y-axis represents IL8R mRNA/beta actin mRNA (with control set to 1.0). IL-8R mRNA was expressed as a proportion of a housekeeping gene (β-actin mRNA).

FIG. 5 is a bar graph showing the effects of a composition embodiment, Composition A (CTP-5), a plant extract, grape pomace extract (GPE), and the combination of the two (CTP-5+GPE) on zymosan-mediated expression of reactive oxygen species in rat neutrophils following exposure to the diet treatments for 6 days. The y-axis represents free radicals (nanomolar). DCF=dichlorofluorescein.

FIG. 6 is a bar graph showing the effects of a composition embodiment, Composition A (CTP-5), a plant extract, grape pomace extract (GPE), and the combination of the two (CTP-5+GPE) on zymosan-mediated expression of reactive oxygen species in rat neutrophils following exposure to the diet treatments for 28 days. The y-axis represents free radicals (picomolar). DCF=dichlorofluorescein.

FIG. 7 is a bar graph showing the effects of a composition embodiment, Composition A (CTP-5), a plant extract, grape pomace extract (GPE), and the combination of the two (CTP-5+GPE) on serum concentrations of C-reactive protein (CRP) in rats following feeding for 6 days. The y-axis is CRP (pg/mL).

FIG. 8 is a bar graph illustrating the amount of L-selectin isolated from neutrophils taken from CTP-5 fed and control fed Beagle dogs (P=0.004; Day 28) wherein L-selectin mRNA is expressed as a proportion of RPL-19 mRNA.

FIG. 9 is a bar graph illustrating the amount of IL-8R isolated from neutrophils taken from CTP-5 fed and control fed Beagle dogs (P=0.029; Day 28), wherein IL-8R mRNA is expressed as a proportion of RPL-19 mRNA.

FIG. 10 is a bar graph illustrating the plasma IL-6 levels from CTP-5 fed and control fed Beagle dogs (P>0.05; Day 14 and Day 28).

FIG. 11 is a bar graph illustrating the plasma IL-4 concentrations from CTP-5 fed and control fed Beagle dogs (P>0.05).

FIG. 12 is a bar graph illustrating the plasma C-reactive protein (CRP) concentrations from CTP-5 fed and control fed Beagle dogs (P>0.05).

FIG. 13 is a bar graph illustrating the plasma interferon-γ (IFN-γ) concentrations from CTP-5 fed and control fed Beagle dogs (P<0.05; Day 14 and Day 28).

FIG. 14 is a graph illustrating concentrations of Aspergillus fumigatus DNA in the plasma of horses.

FIG. 15 is a bar graph illustrating neutrophil interleukin-8 receptor mRNA expression, wherein IL-8R mRNA is expressed as a proportion of RPL-19 mRNA.

FIG. 16 is a bar graph illustrating neutrophil phagocytosis of Streptococcus equi in control- and Composition A-fed animals; two ratios of neutrophil:S. equi were assessed: 30:1 and 60:1.

FIG. 17 is a bar graph illustrating the neutrophil count (million cells/mL) results for male dairy cows treated with four different treatments: a control (“CON”); a composition comprising mineral clay, silica, and beta-glucan supplement (“TRT1”); a direct-fed microbial (“TRT2”); and a composition comprising a combination of mineral clay, silica, beta-glucan supplement, and a direct-fed microbial (“TRT3”).

FIG. 18 is a bar graph illustrating the percent neutrophils observed for male dairy cows treated with the four treatments described for FIG. 17.

FIG. 19 is a bar graph illustrating neutrophil:lymphocyte ratios observed for male dairy cows treated with the four treatments described in FIG. 17.

FIG. 20 is a table illustrating exemplary dose ranges of disclosed exemplary embodiments of the composition and/or combination for various growth stages.

### DETAILED DESCRIPTION

### I. Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Antimicrobial: An agent that kills and/or inhibits the growth of microorganisms. As used herein, antimicrobials include antibiotics, antifungals, antivirals, and antiparasitics including anticoccidials, or combinations thereof.

Administering: Providing a combination, composition, or component disclosed herein by any route to an animal. In some embodiments, administration can refer to oral administration.

Animal: This term can include, but is not limited to, companion animals, utility animals, and feed animals. In some embodiments, an animal can be a companion animal species that is kept as a pet, or an animal species that is raised for human consumption. Exemplary animals are provided herein.

Binding agent or binder: A material or substance that is used to hold or draw together other materials to form a cohesive unit.

Companion Animal: A domesticated animal that is kept as a companion or pet.

Companion Animal Food: A food source for companion animals or other animals that are not feed animals or utility animals.

Binding agent or binder: A material or substance that is used to hold or draw together other materials to form a cohesive unit.

Co-administration: Administering two or more combinations, compositions, or components simultaneously, contemporaneously, or sequentially in any order to a subject to provide overlapping periods of time in which the subject is experiencing effects (e.g., beneficial and/or deleterious effects), from each component. In some embodiments, one or more of the components may be a beneficial or therapeutic agent. Components may be combined into a single composition or dosage form, or they may be administered as separate components either simultaneously or sequentially in any order. When administered sequentially, the two or more components are administered within an effective period of time to provide overlapping periods of time in which the subject experiences effects from each component.

Combination: A combination includes two or more compositions or components that are administered such that the effective time period of the first composition or component overlaps with the effective time period of the second and subsequent compositions or components. A combination may be a composition comprising the components, a composition comprising one or more components and another separate component (or components), or it may be two or more individual components administered substantially simultaneously or sequentially in any order. In an exemplary embodiment of a combination comprising four components, the effective time period of the first component administered may overlap with the effective time periods of the second, third and fourth components, but the effective time periods of the second, third and fourth components independently may or may not overlap with one another. In another exemplary embodiment of a combination comprising four components, the effective time period of the first component administered overlaps with the effective time period of the second component, but not that of the third or fourth; the effective time period of the second component overlaps with those of the first and third components; and the effective time period of the fourth component overlaps with that of the third component only.

Direct-Fed Microbial: A product that can contain live (viable) microorganisms, such as bacteria and/or yeast, which can beneficially affect an animal, such as by improving its intestinal microbial balance.

Effective Amount: A quantity or concentration of a specified combination, composition, or compositional component sufficient to achieve a desired effect in an animal. The therapeutically effective amount may depend at least in part on the species of animal being treated, the size of the animal, and/or the nature of the desired effect.

Excipient or carrier: A physiologically inert substance that can be used as an additive in (or with) a combination, composition, or component as disclosed herein. As used herein, an excipient or carrier may be incorporated within particles of a combination, composition, or component or it may be physically mixed with particles of a combination, composition, or component. An excipient or carrier can be used, for example, to dilute an active agent and/or to modify properties of a combination or composition. Examples of excipients and carriers include, but are not limited to, calcium carbonate, polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

Feed Animal: An animal that is raised for human consumption.

Feedstuff: A food source for utility animals or feed animals. In some embodiments, the term “feedstuff” includes, but is not limited to, solid and liquid animal feeds (e.g., a feed ration), supplements (e.g., a mineral supplement), water, and feed additive carriers (e.g., molasses).

Mannans: A class of polysaccharides including the sugar mannose. The mannan family includes pure mannans (i.e., the polymer backbone consists of mannose monomers), glucomannans (the polymer backbone comprises mannose and glucose), and galactomannans (mannans or glucomannans in which single galactose residues are linked to the polymer backbone). Mannans are found in cell walls of some plant species and yeasts.

Mineral Clay: The term “mineral clay” can refer to hydrous aluminum silicates in some embodiments. In some embodiments, mineral clays can include minor amounts of impurities, such as potassium, sodium, calcium, magnesium, and/or iron. In yet additional embodiments, mineral clays can have a two-layer sheet structure including tetrahedral silicate sheets and octahedral hydroxide sheets or a three-layer structure including a hydroxide sheet between two silicate sheets.

Plant Extract: Polar and non-polar substances that are recovered from plant material via an extraction process that may include a broad range of solvents varying in polarity. Solvents used for such purposes may include, but are not limited to, water, chloroform, hexane, ethyl acetate and ethanol. In some embodiments, a plant extract functions as a biologically-active feed/food ingredient that benefits animal health and well-being.

Polyphenols: A structural class of natural, synthetic, or semisynthetic organic chemicals characterized by the presence of plural phenol structural units.

Saponin: A class of chemical compounds that can be naturally occurring or synthetically prepared. In some embodiments, saponins can be amphipathic glycosides grouped, in terms of structure, by their composition. In certain embodiments, a saponin can comprise one or more hydrophilic glycoside moieties combined with a lipophilic triterpene derivative.

Therapeutic agent: An agent that is capable of providing a therapeutic effect, e.g., preventing a disorder, inhibiting a disorder, such as by arresting the development of the disorder or its clinical symptoms, or relieving a disorder by causing regression of the disorder or its clinical symptoms.

Utility Animal: An animal that is raised to produce a product for human use or consumption.

### II. Compositions and Combinations

Disclosed herein are embodiments of compositions and combinations that can be used for feeding animals, such as companion animals, utility animals, or feed animals. In some embodiments, the compositions and combinations can be used for feeding aquatic animal, including, but not limited to, fish, crustaceans, and mollusks. Combination or composition embodiments disclosed herein can comprise, consist essentially of (e.g., composition or combination contains no more than 5%, no more than 4%, no more than 3%, no more than 2%, no more than 1%, or no more than 0.5% of other agents, such as 0.01 to 5%, 0.1 to 5% 1 to 5% or 0.1 to 1%), or consist of two or more compositions disclosed herein. In some embodiments, the combination or composition can consist essentially of any component that does not materially affect the combination, such as mycotoxins, chelators, pathogenic bacteria, pathogenic fungi, or pathogenic viruses. In exemplary embodiments, the combinations can be added to food consumed by companion animals, such as felines and canines. In other exemplary embodiments, the combinations can be added to feedstuff consumed by feed animals, such as livestock, or utility animals. In yet other embodiments, the combination can be provided as a supplement that is fed directly to the animal prior to or after a feedstuff or companion animal food. The present disclosure, however, also contemplates using the disclosed combinations as a supplement for feed, or even as a direct food source, provided to, for example, pigs, domestic fowl (e.g., chicken, turkey, goose, duck, cornish game hen, quail, pheasant, guinea-fowl, ostrich, emu, swan, or pigeon), fish (e.g., salmon, trout, tilapia, and the like), reptiles, crustaceans, rabbits, sheep, goats, cows, deer, bison, buffalos, alpacas, horses, donkeys, or llamas.

Various composition embodiments are disclosed herein. In some embodiments, a composition used in the combination embodiments disclosed herein can comprise, consist essentially of, or consist of any one or more of a β-glucan (e.g., β-1,3 (4)glucan), silica, mineral clay, mannans, or an endoglucanohydrolase (e.g., β-1,3 (4)-endoglucanohydrolase). In other embodiments, a composition can comprise, consist essentially of, or consist of yucca, quillaja, a direct-fed microbial, or combinations thereof. In some embodiments, a composition can comprise, consist essentially of, or consist of one or more vitamin D species. In other embodiments, a composition can comprise, consist essentially of, or consist of a chromium compound. In yet other embodiments, a composition can comprise, consist essentially of, or consist of a plant extract. In some embodiments, the combinations disclosed herein can comprise, consist essentially of, or consist of two or more components and/or compositions disclosed herein. In some embodiments, the combinations disclosed herein also can comprise an animal food, such as a feedstuff or a companion animal food. In exemplary embodiments, the combinations can comprise, consist essentially of, or consist of β-glucan, silica, mineral clay, mannans, β-1,3 (4)-endoglucanohydrolase, yucca, quillaja, a direct-fed microbial, a plant extract, a vitamin D compound, a chromium compound, or any combination thereof. Compositional components disclosed herein can be obtained or made from a variety of sources, which are disclosed herein. Exemplary compositional components are discussed in more detail below.

Some compositions disclosed herein can comprise silica obtained from a source including, but not limited to, sand, quartz, diatomaceous earth, and synthetic silica. In some embodiments, the silica can be synthetic silica that is structurally distinct from naturally occurring silica. In certain embodiments, the mannans can comprise, consist essentially of, or consist of glucomannan. In some embodiments, the β-glucan can include soluble and/or insoluble β-glucan, such as (1,3/1,4) β-glucan (β-1,3 (4) glucan), (1,3/1,6) β-glucan, or a combination thereof.

Suitable plant materials from which a plant extract can be obtained include, but are not limited to, apples, blackberries, black chokeberries, black currants, black elderberries, blueberries, cherries, cranberries, grapes, green tea, hops, onions, quillaja, plums, pomegranates, raspberries, strawberries, and yucca.

Examples of yucca that can be used in the disclosed compositions and combinations include, but are not limited to, Yucca aloifolia, Yucca angustissima, Yucca arkansana, Yucca baccata, Yucca baileyi, Yucca brevifolia, Yucca campestris, Yucca capensis, Yucca carnerosana, Yucca cernua, Yucca coahuilensis, Yucca constricta, Yucca decipiens, Yucca declinata, Yucca desmetiana, Yucca elata, Yucca endlichiana, Yucca faxoniana, Yucca filamentosa, Yucca filifera, Yucca flaccida, Yucca gigantean, Yucca glauca, Yucca gloriosa, Yucca grandiflora, Yucca harrimaniae, Yucca intermedia, Yucca jaliscensis, Yucca lacandonica, Yucca linearifolia, Yucca luminosa, Yucca madrensis, Yucca mixtecana, Yucca necopina, Yucca neomexicana, Yucca pallida, Yucca periculosa, Yucca potosina, Yucca queretaroensis, Yucca reverchonii, Yucca rostrata, Yucca rupicola, Yucca schidigera, Yucca schottii, Yucca sterilis, Yucca tenuistyla, Yucca thompsoniana, Yucca treculeana, Yucca utahensis, or Yucca valida. In certain disclosed embodiments the yucca component is Yucca schidigera.

Examples of quillaja that can be used in the disclosed combination include, but are not limited to, Quillaja brasiliensis, Quillaja lanceolata, Quillaja lancifolia, Quillaja molinae, Quillaja petiolaris, Quillaja poeppigii, Quillaja saponaria, Quillaja sellowiana, or Quillaja smegmadermos. In particular disclosed embodiments the quillaja is Quillaja saponaria.

Examples of direct-fed microbials can include Bacillus species, such as B. alcalophilus, B. alvei, B. aminovorans, B. amyloliquefaciens, B. aneurinolyticus, B. anthracia, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. galliciensis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis, B. weihenstephanensis, or combinations thereof. In particular disclosed working embodiments, the Bacillus is Bacillus coagulans (or B. coagulans). A person of ordinary skill in the art will appreciate that, as used herein, the bacterial name may refer to the bacteria, or to a compound or compounds obtained from that bacteria. Methods of obtaining compounds from bacteria are well known in the art.

Vitamin D compounds that can be used in the compositions and combinations disclosed herein can be selected from vitamin D3, 25-hydroxy vitamin D3, 25-dihydroxy vitamin D3, and combinations thereof. In exemplary embodiments, 25-hydroxy vitamin D3 (or a composition thereof) can be used.

Chromium compounds that can be used in the compositions and/or combinations disclosed herein include any chromium compound suitable for feed, food, pharmaceutical or veterinary use. The chromium compound may be a chromium(III) compound. The chromium compound may be a chromium compound, a chromium salt, of a combination thereof. Exemplary chromium compounds include, but are not limited to, chromium organic acid compounds, such as chromium picolinate, chromic tripicolinate, chromium nicotinate, chromic polynicotinate, chromium acetate, or chromium propionate, or chromium amino acid compounds, such as chromium histidinate, chromium nicotinate-glycinate, chromium glycinate, chromium aspartate, or chromium phenylalanine; chromium halides, such as chromium chloride, chromium bromide, chromium iodine or chromium fluoride; chromium yeast; chromium carbonate; or a combination thereof. Additional information concerning chromium compounds can be found in U.S. Patent Publication No. 2010/0178362, which is incorporated herein by reference.

In some embodiments, combinations disclosed herein can comprise a composition, such as a primary composition comprising, consisting essentially of, or consisting of mineral clay and silica, β-glucan, or a combination thereof. In some embodiments, the primary composition can comprise 1-40 wt % silica, 0.5-30 wt % β-glucans and mannans, 35-92 wt % mineral clay, such as 1-40 wt % silica, 1-30 wt % β-glucans and mannans, 35-92 wt % mineral clay, and can be combined with any one or more of yucca, quillaja, a direct-fed microbial, a vitamin D3 compound, a chromium compound, or a plant extract (or a composition of any of these components). Another combination embodiment can comprise a composition comprising, consisting essentially of, or consisting of 1-40 wt % silica, 0.5-25 wt % glucan and mannans, and 40-92 wt % mineral clay, such as 1-40 wt % silica, 1-25 wt % glucan and mannans, and 40-92 wt % mineral clay, and any one or more of yucca, quillaja, a direct-fed microbial, a vitamin D3 compound, a chromium compound, or a plant extract (or a composition of any of these components). Other combination embodiments can comprise a composition comprising, consisting essentially of, or consisting of 5-40 wt % silica, 0.5-15 wt % β-glucans and mannans, 40-80 wt % mineral clay, such as 5-40 wt % silica, 2-15 wt % β-glucans and mannans, 40-80 wt % mineral clay, and any one or more of yucca, quillaja, a direct-fed microbial, a vitamin D3 compound, a chromium compound, or a plant extract (or a composition of any of these components). In some embodiments, combination embodiments can comprise a composition comprising, consisting essentially of, or consisting of 20-40 wt % silica, 0.5-10 wt % β-glucans and mannans, 50-70 wt % mineral clay, such as 20-40 wt % silica, 2-10 wt % β-glucans and mannans, 50-70 wt % mineral clay, and any one or more of yucca, quillaja, a direct-fed microbial, a vitamin D compound, a chromium compound, or a plant extract (or a composition of any of these components). Yet other combination embodiments can comprise a composition comprising, consisting essentially of, or consisting of 15-40 wt % silica, greater than zero to 15 wt % glucans, zero to 10 wt % mannans, typically greater than zero to 10 wt % mannans, and 50-81 wt % mineral clay, such as 15-40 wt % silica, 1-15 wt % β-glucans, zero to 10 wt % mannans, and 50-81 wt % mineral clay, and any one or more of yucca, quillaja, a direct-fed microbial, a vitamin D3 compound, a chromium compound, or a plant extract (or a composition of any of these components). In yet other embodiments, combinations embodiments can comprise a composition comprising, consisting essentially of, or consisting of 20-30 wt % silica; 0.5-3.5 wt % β-glucans, such as 1.0-3.5 wt % β-glucans; 0.5-6.0 wt % mannans, such as 1.0-6.0 wt % mannans; 60-75 wt % mineral clay, such as 60-70 wt % mineral clay, and any one or more of yucca, quillaja, a direct-fed microbial, a vitamin D3 compound, a chromium compound, or a plant extract (or a composition of any of these components).

In any of the above embodiments, the combination or composition can further comprise an endoglucanohydrolase, such as β-1,3 (4)-endoglucanohydrolase. As used herein, weight % for endoglucanohydrolase is based on a 70,000 unit/gram endoglucanohydrolase product. Certain combination embodiments can be formulated to comprise, consist essentially of, or consist of a composition disclosed above, or any one or more of the components disclosed above, and further comprising from at least 0.05 wt % endoglucanohydrolase to 5 wt % endoglucanohydrolase, such as from 0.05-3 wt % β-1,3 (4)-endoglucanohydrolase. In some embodiments, a composition used in the combinations disclosed herein can comprise 0.2-3 wt %, β-1,3 (4)-endoglucanohydrolase, 20-40 wt % silica, 1-10 wt % β-glucans and mannans, and 50-70 wt % mineral clay. In another embodiment, the composition consists essentially of 0.1-3 wt %, β-1,3 (4)-endoglucanohydrolase, 20-40 wt % diatomaceous earth, 2-20 wt % β-glucan and glucomannans, and 50-70 wt % mineral clay.

In one embodiment, the primary composition can comprise 0.1-3 wt % β-1,3 (4)-endoglucanohydrolase, such as 0.1-1 wt % β-1,3 (4)-endoglucanohydrolase; 20-40 wt % silica, such as diatomaceous earth; 5-20 wt % yeast cell wall or an extract thereof; and 40-80 wt % mineral clay. In another embodiment, the primary composition can comprise 0.1-0.5 wt % β-1,3 (4)-endoglucanohydrolase, 20-30 wt % silica, such as diatomaceous earth, 5-15 wt % yeast cell wall or an extract thereof, and 60-70 wt % mineral clay. In still another embodiment, the primary composition can comprise 0.1-0.3 wt % β-1,3 (4)-endoglucano-hydrolase, 24-25 wt % silica, such as diatomaceous earth, 10-11 wt % yeast cell wall or an extract thereof, and 63-64 wt % mineral clay.

In an independent embodiment, β-1,3 (4)-endoglucanohydrolase, silica, such as diatomaceous earth, yeast cell wall or an extract thereof and mineral clay can be combined at 0.05-3%, 1-40%, 1-20% and 37-92%, respectively, to form a composition suitable for use in a combination. In another independent embodiment, β-1,3 (4)-endoglucanohydrolase, silica, such as diatomaceous earth, yeast cell wall or an extract thereof and mineral clay can be combined at 0.1-3%, 5-40%, 2-15% and 40-80%, respectively, to form a composition that can be used to make a combination disclosed herein. In yet another independent embodiment, β-1,3 (4)-endoglucanohydrolase, silica, such as diatomaceous earth, yeast cell wall or an extract thereof and mineral clay can be combined at 0.2-3%, 20-40%, 4-15% and 50-65%, respectively, to make a composition that can be used to make a disclosed combination.

Certain combination embodiments, such as those disclosed above, can further comprise, consist essentially of, or consist of yucca, quillaja, or a combination or composition thereof. In exemplary embodiments, the disclosed combinations can comprise a composition comprising 85% Quillaja saponaria and 15% Yucca schidigera.

In particular disclosed embodiments, combinations can further comprise a direct-fed microbial, such as a Bacillus species. In some embodiments, the Bacillus species can be provided in a composition comprising, consisting essentially of, or consisting of yucca and quillaja. In exemplary embodiments, the Bacillus species is B. coagulans.

In yet other embodiments, the combinations can comprise, consist essentially of, or consist of any one or more of the compositions disclosed above and further comprising 25-hydroxy vitamin D3, a plant extract, or a combination thereof.

In certain embodiments, the combinations can comprise, consist essentially of, or consist of one or more of the compositions disclosed above and further comprise one or more chromium compounds. In some embodiments, the chromium compound(s) comprise a chromium (III) compound. Exemplary chromium compounds include, but are not limited to, chromium organic acid compounds, such as chromium picolinate, chromic tripicolinate, chromium nicotinate, chromic polynicotinate, chromium acetate, or chromium propionate, or chromium amino acid compounds, such as chromium histidinate, chromium nicotinate-glycinate, chromium glycinate, chromium aspartate, or chromium phenylalanine; chromium halides, such as chromium chloride, chromium bromide, chromium iodine or chromium fluoride; chromium yeast; chromium carbonate; chromium nitrate; chromium sulfate; chromium phosphate; chromium nitrite; or a combination thereof. The amount of chromium compound may be sufficient to provide a daily dose of from 0.001 milligram to 5000 milligrams of a total chromium compound per kilogram body weight, such as from 0.01 milligram total chromium compound to 1000 milligrams per kilogram body weight, from 0.1 milligram to 100 milligrams per kilogram body weight, from 0.5 milligram to 25 milligrams per kilogram body weight, or from 1 milligram to 10 milligrams per kilogram body weight.

In some embodiments, the amount of chromium compound in the combination is selected to provide a sufficient amount of chromium to the subject. The sufficient amount of chromium may be from 0.5 μg per day to 10,000 μg per day or more, such as from 5 μg to 10,000 μg/day, from 25 μg to 10,000 μg per day, from 50 μg to 10,000 μg per day, from 100 μg to 10,000 μg per day, from 200 μg to 10,000 μg per day, from 300 μg to 10,000 μg per day, from 400 μg to 10,000 μg per day, from 500 μg to 10,000 μg per day, from 750 μg to 10,000 μg per day, from 1,000 μg to 10,000 μg per day, from 1500 μg to 10,000 μg per day, from 2,000 μg to 10,000 μg per day, from 2500 μg to 10,000 μg per day, from 3000 μg to 10,000 μg per day, from 3500 μg to 10,000 μg per day, from 4000 μg to 10,000 μg per day, from 4500 μg to 10,000 μg per day, from 5,000 μg to 10,000 μg per day, from 6000 μg to 10,000 μg per day, from 7000 μg to 10,000 μg per day, from 8000 μg to 10,000 μg per day, from 9000 μg to 10,000 μg per day or more chromium/day.

For example, the amount of chromium provided by the chromium compound(s) to inhibit the onset of insulin resistance may be from 0.5 μg to 30 μg/kg/day, such as from 2 μg to 30 μg/kg/day, from 4 μg to 15 μg/kg/day, from 5 μg to 15 μg/kg/day, or from 8 μg to 15 μg/kg/day.

In some embodiments, the combinations disclosed herein can include one or more components in addition to the compositional components disclosed above. Additional components may be used for any desired purpose, such as a substantially biologically inert material added, for example, as a filler, or to provide a desired beneficial effect. For example, the combinations can further comprise a carbonate (including a metal carbonate such as calcium carbonate), sorbic acid or a salt thereof (such as potassium sorbate, sodium sorbate, ammonium sorbate, or a combination thereof), kelp, a vitamin (such as vitamin A, vitamin B-1, vitamin B-2, vitamin B-3, vitamin B-5, vitamin B-6, vitamin B-12, vitamin C, vitamin D, vitamin E, vitamin K, or a combination thereof, typically a niacin supplement or vitamin B-12 supplement, biotin, d-calcium pantothenate, choline chloride, thiamine mononitrate, pyridoxine hydrochloride, menadione dimethylpyrimidinol bisulfite, riboflavin-5-phosphate, or folic acid), soybean oil, calcium aluminosilicate, rice hulls, an oil (such as mineral oil, corn oil, soybean oil, or a combination thereof), a trace mineral (such as, but not limited to, chloride, fluoride, iodide, chromium, copper, zinc, iron, magnesium, manganese, molybdenum, phosphorus, potassium, sodium, sulfur, selenium, or a combination thereof), a micro tracer (such as iron particles coated with a dye), yeast (such as yeast culture, active yeast, a live yeast, a dead yeast, yeast extract, or a combination thereof), a preservative (such as benzoic acid or a salt thereof, e.g. sodium benzoate; lactic acid or a salt thereof, e.g. sodium lactate, potassium lactate or calcium lactate; propionic acid or a salt thereof, e.g. sodium propionate; ascorbic acid or a salt thereof, e.g. sodium ascorbate; gallic acid or a salt thereof e.g. sodium gallate; sulfur dioxide and/or sulfites; nitrites; nitrates; choline, or a salt thereof, such as an anion salt of choline, e.g. choline halide, such as chloride, bromide, iodide, fluoride, or choline hydroxide; or any combination thereof), or any combination thereof. In some embodiments, the combination can comprise a binder selected from acacia, alginic acid, carboxymethylcellulose, sodium compressible sugar, ethylcellulose gelatin, liquid glucose, methylcellulose, povidone, pregelatinized starch, or combinations thereof. In some embodiments, the combination can comprise an excipient selected from calcium carbonate, polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, lactose, or combinations thereof.

In an exemplary embodiment, the combination can comprise a primary composition comprising silica, mineral clay, β-glucan, and optionally mannans, or any combination thereof; and any one or more of Quillaja saponaria, Yucca schidigera, B. coagulans, 25-hydroxy vitamin D3, or chromium compound.

In some embodiments, the compositions and combinations also can comprise an antimicrobial, an antibiotic, an anticoccidial agent, a vaccine, and/or combinations of such components.

Suitable antimicrobials and/or antibiotics include, but are not limited to, Virginiamycin, Bacitracin MD, Zinc Bacitracin, Tylosin, Lincomycin, Flavomycin, Terramycin, Neo-Terramycin, or combinations thereof. In yet additional embodiments, the antimicrobial or antibiotic can be selected from penicillin, tetracycline, ceftiofur, florfenicol, tilmicosin, enrofloxacin, and tulathromycin, procaine penicillin, benzathine penicillin, ampicillin, amoxicillin, spectinomycin, dihydrostreptomycin, chlortetracycline, gentamicin, sulphadimidine, trimethoprim, oxytetracycline, erythromycin, norfloxacin and combinations thereof.

Suitable anticoccidial agents include, but are not limited to, ionophores and chemical anticoccidial products. Ionophores can include, but are not limited to, Monensin, Salinomycin, Lasalocid, Narasin, Maduramicin, Semduramicin, Laidlomycin, or combinations thereof.

Chemical anticoccidial products can include, but are not limited to, Nicarbazin, Maxiban, Diclazuril, Toltrazuril, Robenidine, Stenorol, Clopidol, Decoquinate, DOT (zoalene), Amprolium, or combinations thereof.

Suitable vaccines can be selected from live coccidiosis vaccines, such as COCCIVAC (e.g., a composition comprising live oocysts of Eimeria acervulina, Eimeria mivati, Eimeria maxima, Eimeria mitis, Eimeria tenella, Eimeria necatrix, Eimeria praecox, Eimeria brunetti, Eimeria hagani, or combinations thereof), LivaCox (a composition comprising 300-500 live sporulated oocysts of each attenuated line of Eimeria acervulina, E. maxima and E. tenella in a 1% w/v aqueous solution of Chloramine B), ParaCox (a composition comprising live sporulated oocysts derived from E. acervulina HP, E. brunetti HP, E. maxima CP, E. maxima MFP, E mitis HP, E. necatrix HP, E. praecox HP, E. tenella HP, and combinations thereof), Hatch Pack Cocci III (a composition comprising oocysts derived from Eimeria acervulina, Eimeria maxima, Eimeria tenella, or combinations thereof), INOVOCOX (a composition comprising oocysts derived from Eimeria acervulina, Eimeria maxima, Eimeria tenella, and a sodium chloride solution), IMMUCOX (a composition comprising live oocysts derived from Eimeria acervulina, Eimeria maxima, Eimeria necatrix, Eimeria tenella, and combinations thereof), Advent, or combinations thereof. Vaccines may also comprise live oocysts of the Eimeria genus, for example, Eimeria aurati, Eimeria baueri, Eimeria lepidosirenis, Eimeria leucisci, Eimeria rutile, Eimeria carpelli, Eimeria subepithelialis, Eimeria funduli and/or Eimeria vanasi.

The amount of antimicrobial or antibiotic used is within the amounts stated below but may depend on the particular antimicrobial or antibiotic used as will be understood by a person of ordinary skill in the art. In an independent embodiment, the amount of the antibiotic or antimicrobial that is used can be a therapeutically effective amount that is at an approved or authorized dosage level for a particular antibiotic. In some embodiments, the amount of antibiotic or antimicrobial used can range from greater than 0 ppm to 100,000 ppm, such as 0.25 ppm to 5,000 ppm, or 0.5 ppm to 2,500 ppm, or 0.75 ppm to 2,000 ppm, or 1 ppm to 1,500 ppm, or 5 ppm to 1,000 ppm, or 10 ppm to 500 ppm, or 25 ppm to 300 ppm. In yet additional embodiments, the amount of antibiotic or antimicrobial used can range from greater than 0 mg/kg of body weight to 100,000 mg/kg of body weight, such as 0.5 mg/kg to 2,500 mg/kg, or 1 mg/kg to 1,500 mg/kg, or 5 mg/kg to 1,000 mg/kg, or 10 mg/kg to 500 mg/kg m, or 25 mg/kg to 300 mg/kg, or 10-20 mg/kg.

In some embodiments, the amount of the antimicrobial or antibiotic that is included in the composition can range from at least 1 g/ton of feed to 230 g/ton of feed (or at least 1.1 ppm to 256 ppm), such as at least 1 g/ton of feed to 220 g/ton of feed (or at least 1.1 ppm to 243 ppm), at least 1 g/ton of feed to 100 g/ton of feed (or at least 1.1 ppm to 110 ppm), at least 1 g/ton of feed to 50 g/ton of feed (or at least 1.1 ppm to 55 ppm), or at least 1 g/ton of feed to 10 g/ton of feed (or at least 1.1 ppm to 11 ppm). Particular antimicrobials or antibiotics that can be used, and dosage amounts of such antimicrobials and antibiotics include, but are not limited to, the following: Virginiamycin in an amount ranging from 5 g/ton of feed to 25 g/ton of feed (or 5 ppm to 27 ppm, such as 22 ppm); Bacitracin MD in an amount ranging from 40 g/ton of feed to 220 g/ton of feed (or 44 ppm to 242 ppm, or 50 ppm to 250 ppm in some other embodiments); Zinc Bacitracin in an amount ranging from 40 g/ton of feed to 220 g/ton of feed (or 44 ppm to 242 ppm); Tylosin in an amount ranging from 1 g/ton of feed to 1000 g/ton of feed (or 1 ppm to 1100 ppm); Lincomycin in an amount ranging from 1 g/ton of feed to 5 g/ton of feed (or 1 ppm to 6 ppm); Flavomycin in an amount ranging from 1 g/ton of feed to 5 g/ton of feed (or 1 ppm to 6 ppm); or combinations thereof.

The amount of anticoccidial agent, as will be understood by a person of ordinary skill in the art (e.g., a veterinarian), can be selected depending on the particular anticoccidial agent used. In some embodiments, the amount of anticoccidial agent used can be a therapeutically effective amount for a particular animal species. In some embodiments, the amount of anticoccidial agent used can range from greater than 0 ppm to 100,000 ppm, such as 0.25 ppm to 5,000 ppm, or 0.5 ppm to 2,500 ppm, or 0.75 ppm to 2,000 ppm, or 1 ppm to 1,500 ppm, or 5 ppm to 1,000 ppm, or 10 ppm to 500 ppm, or 25 ppm to 300 ppm. In yet additional embodiments, the amount of antibiotic or antimicrobial used can range from greater than 0 mg/kg of body weight to 100,000 mg/kg of body weight, such as 0.5 mg/kg to 2,500 mg/kg, or 1 mg/kg to 1,500 mg/kg, or 5 mg/kg to 1,000 mg/kg, or 10 mg/kg to 500 mg/kg m, or 25 mg/kg to 300 mg/kg, or 10-20 mg/kg.

In some embodiments the composition and/or combination further comprises a vitamin, a trace mineral, a bulking agent, a carrier, a colorant, a taste enhancer, or any combination thereof. In other embodiments the combination and/or composition further comprises corn, soybean meal, wheat, barley, rye, canola, corn oil, limestone, salt, distillers dried grains with solubles (DDGS), dicalcium phosphate, sodium sesquicarbonate, methionine source, lysine source, L-threonine, choline, or any combination thereof.

In some embodiments, the composition and/or combination includes a coating agent. The amount of coating agent may be from zero to 10% or more by weight, such as from greater than zero to 10% or from 2% to 10% by weight. The coating agent is a material selected to, for example, facilitate adhering some or all of the components of the composition and/or combination together, to a foodstuff, or both. The coating agent also may facilitate maintaining adherence of the composition and/or combination together or to a foodstuff in an aquatic environment to facilitate administration to aquatic species. The material is also preferably palatable and edible by aquatic animals.

In some embodiments the coating agent is an oil. For example, the oil may be selected from corn oil, coconut oil, linseed oil cottonseed oil, olive oil, peanut oil, palm oil, canola oil, safflower oil, soy oil, sunflower oil, Naskole oil, or any combination thereof. In some embodiments, the coating agent is a syrup. For example, the syrup may be selected from molasses, sorghum, sugar syrup, honey, or any combination thereof. Combinations of oils and syrups also may be used.

In some embodiments, the composition and/or combination may be used to replace or supplement animal feedstuffs. In some embodiments, the feedstuff is a commercial feedstuff. In particular embodiments, the feedstuff was manufactured by Raanan Fish Meal. The feed may be formulated as sinking extruded pellets #4932S0 at sizes of 2-4 mm Certain particular feed embodiments comprised 45.0% protein, 12.0% fat, 3.0% carbohydrates, 9% ash, and 9.8% moisture. In other particular embodiments, the feedstuff was manufactured by Zemach Feed Mill. The feed may be formulated as floating extruded pellets #4662 at sizes of 2-4 mm Certain particular embodiments comprised 35.0% protein, 3.5% fat, 14.0% carbohydrates, 8.0% ash, and 10.0% moisture. In other particular embodiments the feed used was manufactured by Zemach Feed Mill, and was based on floating extruded pellets #4212 at a size of 4 mm Certain particular embodiments comprised 30.0% protein, 5.0% fat, 4.5% carbohydrates, 8.0% ash, and 10.0% moisture. In some embodiments, the composition or one or more of the components of the combination is coated on the feedstuffs using a coating agent.

In some embodiments, the composition and/or combination includes additional components. Additional components may be used for any desired purpose, such as a substantially biologically inert material added, for example, as a filler, or to provide a desired beneficial effect. Alternatively or in addition, adjuvants and/or therapeutic agents also may be included in the composition and/or combination. For example, composition and/or combination may include, without limitation, a carbonate (including a metal carbonate such as calcium carbonate), kelp, a vitamin (such as a niacin supplement or vitamin B-12 supplement), biotin, d-calcium pantothenate, choline chloride, thiamine mononitrate, pyridoxine hydrochloride, menadione dimethylpyrimidinol bisulfite, riboflavin-5-phosphate, folic acid, soybean oil, calcium aluminosilicate, rice hulls, algae, mineral oil, or any combination thereof. The algae may be a blue-green algae (cyanobacteria), a diatom (bacillariophyta), a stonewort algae (charophyta), a green algae (chlorophyta), a golden algae (chrysophyta), a dinoflagellate (dinophyta), a brown algae (phaeophyta) or a red algae (rhodophyta). In some embodiments, the algae is a chlorophyta, and may be an algae from the genus Chlorella, including, but not limited to, Chlorella vulgaris, Chlorella angustoellipsoidea, Chlorella botryoides, Chlorella capsulata, Chlorella ellipsoidea, Chlorella emersonii, Chlorella fusca, Chlorella homosphaera, Chlorella luteo-v iridis, Chlorella marina, Chlorella miniata, Chlorella minutissima, Chlorella mirabilis, Chlorella ovalis, Chlorella parasitica, Chlorella peruviana, Chlorella rugosa, Chlorella saccharophila, Chlorella salina, Chlorella spaerckii, Chlorella sphaerica, Chlorella stigmatophora, Chlorella subsphaerica, Chlorella trebouxioides, or a combination thereof. In other embodiments, the algae is a cyanobacteria, such as Arthrospira platensis or Arthrospira maxima (spirulina). Other algae include, but are not limited to, algae of the genus Pediastrum, such as Pediastrum dupl, Pediastrum boryanum, or a combination thereof, algae of the genus Botryococcus, such as Botryococcus braunii, algae of the genus Porphyra, such as Porphyra dioica, Porphyra linearis, Porphyra lucasii, Porphyra mumfordii, Porphyra purpurea, Porphyra umbilicalis, or a combination thereof.

In some embodiments, a composition and/or combination disclosed herein does not comprise a peroxide compound.

In some embodiments, a composition and/or combination disclosed herein does not comprise hydrogen peroxide.

In some embodiments, a composition and/or combination disclosed herein does not comprise carbamide peroxide.

In some embodiments, a composition and/or combination disclosed herein does not comprise urea.

In some embodiments, a composition and/or combination disclosed herein does not comprise hydrogen peroxide and urea.

More information concerning the disclosed compositions and combinations, and the methods for making and using such compositions and combinations, can be found in U.S. Pat. Nos. 8,142,798 and 8,236,303; U.S. patent application Ser. Nos. 14/606,862, 14/699,740, 15/234,971, and 15/359,342; and PCT application No. PCT/US2016/051080, all of which are incorporated herein by reference in their entirety.

### III. Methods of Making the Compositions and Combinations

Also disclosed herein are methods of making the compositions and combinations disclosed herein. In particular disclosed embodiments, compositions for use in the disclosed combinations can be made by combining two or more compositional components disclosed above. In some embodiments, two or more compositions can be mixed to form a combination as disclosed herein. The compositions can be combined in a ratio or proportion suitable for administration to an animal. In some embodiments, the compositions can be combined by mixing one or more compositional components together and the combinations of such compositions can be made by combining these compositions. In some embodiments, the compositional components and compositions can be mixed as solids, liquids, or combinations thereof.

In some embodiments, certain components of the disclosed compositions and combinations can be prepared by methods commonly known in the art or can be obtained from commercial sources. In some embodiments, some components of the disclosed compositions and combinations can be present in the environment or can be modified from a naturally occurring state or synthesized so as to form a non-naturally occurring compound.

In any of the above disclosed combination embodiments, the silica can be obtained from diatomaceous earth. In some embodiments, diatomaceous earth is a commercially-available product comprised primarily of silica (SiO₂) and with its remaining components not assayed but consisting primarily of ash (minerals) as defined by the Association of Analytical Chemists (AOAC, 2002). In yet other embodiments, the silica can be modified from its naturally occurring state and/or prepared synthetically to provide synthesized silica.

In some embodiments, the β-glucans can be obtained from yeast, or other materials, such as fungi, algae, or the like. In any of the above combination embodiments, the mannans can comprise glucomannan.

The mineral clays (aluminosilicates) used in particular composition embodiments that can be used to make combination embodiments disclosed herein can be fulfilled by any of a variety of commercially-available clays including, but not limited to, montmorillonite clay, bentonite and zeolite.

In some embodiments, β-glucans and mannans can be obtained from plant cell walls, yeast (e.g., Saccharomyces cerevisiae, Candida utilis), certain fungi (e.g., mushrooms), algae, bacteria, or combinations thereof. One exemplary commercial source of β-glucans and mannans (e.g., β-1,3 (4) glucan and glucomannan) is a yeast cell wall or an extract thereof derived from inactivated yeast (Saccharomyces cerevisiae). The yeast cell wall or an extract thereof may have a composition comprising 0-15% moisture and 85-100% dry matter, such as 0-8% moisture, and 92-100% dry matter. The dry matter may comprise 10-65% protein, 0-25% fats, 0-3% phosphorus, 5-30% β-glucan, 0-35% mannans, and 0-15% ash, such as 10-55% protein, 0-25% fats, 0-2% phosphorus, 10-30% β-glucan, 0-25% or 5-35% mannans, and 0-5% ash. In particular examples disclosed herein, the yeast cell wall or an extract thereof can comprise the following composition: 2-6% moisture, 94-98% dry matter, 20-45% proteins, 0.1-22% fats, 0.5-3% phosphorus, 10-25% mannans, 14-30% β-1,3 (4) glucan, and 3-5% ash. In an independent embodiment, a commercial source of β-1,3 (4) glucan and glucomannan derived from primary inactivated yeast (Saccharomyces cerevisiae) with the following chemical composition can be used: moisture 2-5%; proteins 40-50%; fats 3-8%; phosphorus 0-2%; mannans 10-16%; 13-1,344) glucan 10-20%; and ash 2-12%.

In another independent embodiment, the yeast cell wall or an extract thereof comprises moisture 1-7% and dry matter 93-99%, and the dry matter may comprise 18-28% proteins, 10-17% fats, 0-2% phosphorus, 20-30% mannans, 18-28%13-1,344) glucan, and 2-5% ash.

In an independent embodiment, a commercial source of β-1,3 (4) glucan and glucomannan derived from primary inactivated yeast (Saccharomyces cerevisiae) with the following chemical composition can be used: moisture 3.5-6.5%; proteins 30-42%; fats 0.1-10.5%; phosphorus 0-1.5%; mannans 11-25%; β-1, 3-(4) glucan 15-29%; ash 3-5%; and in some embodiments, the composition can further comprise dry matter 94-98%. In another independent embodiment, the chemical composition can comprise, consist essentially of, or consist of moisture 2-5%; dry matter 94-98%; proteins 14-36%; fats 5-22%; phosphorus 0.5-2%; mannans 15-24%; 13-1, 3-(4) glucan 18-26%; and ash 3-5%.

In some embodiments, β-1,3 (4)-endoglucanohydrolase may be produced from submerged fermentation of a strain of Trichoderma longibrachiatum.

The yucca and quillaja components disclosed herein can include any of the plants species from which the yucca or quillaja is obtained as a whole, or any part of the plant, such as the roots, stem or trunk, bark, leaves, flower, flower stems, or seeds or a combination thereof. These plant parts may be used fresh, or dried, and may be whole, pulverized, mashed chopped up or ground up.

Embodiments of the disclosed plant extracts can be prepared from polyphenol-containing plant material. The plant material also may include non-polyphenol compounds, including polyphenol degradation products, such as gallic acid and trans-caftaric acid. Degradation can occur, for example, through oxidative and/or biological processes. Both the polyphenols and the non-polyphenol compounds may have biological activity. The plant extract may be prepared from a single plant material (e.g., grapes) or from a combination of plant materials. In some embodiments, the plant extract is prepared from a pressed plant material, such as grape pomace, a dried plant material, such as tea, or a combination thereof. Pomace may be obtained substantially immediately post-pressing or as an ensiled product, i.e., pomace collected and stored for up to several months post-pressing. Suitable plants have a plurality of polyphenols and/or other non-polyphenolic compounds, including but not limited to non-polyphenolic organic acids (such as gallic acid and/or trans-caftaric acid), flavanols, gallate esters, flavanodiols, phloroglucinol, pyrogallol, and catechol. In some embodiments, the plant extract is prepared from Pinot noir pomace, Pinot gris pomace, or green tea.

In some embodiments, pressed or dried plant material is ground to a fine powder prior to, or during, extraction. Pressed plant materials may be frozen to facilitate grinding. Polyphenols and other non-polyphenolic compounds may be extracted for administration. For example, polyphenols and other non-polyphenolic compounds may be extracted from the powder using a solution comprising a polar solvent, such as water, an alcohol, an ester, or a combination thereof. In some embodiments, the solution comprises a water-miscible alcohol, ester, or combination thereof, such as a lower alkyl alcohol, lower alkyl ester, or a combination thereof. In some embodiments, the solution is water or an aqueous solution comprising 25-99% solvent, such as 25-95% solvent, 30-80% solvent, or 50-75% solvent, and water. In certain embodiments, the solution is an aqueous solution comprising methanol, ethanol, isopropanol, ethyl acetate, or a combination thereof. The solution may be acidified by addition of an acid. The acid may prevent or minimize oxidative degradation of biologically-active polyphenols and other non-polyphenolic compounds in the extract. The acid may be any suitable acid, such as a mineral acid (e.g., hydrochloric acid), or an organic acid such as citric acid or acetic acid. In some embodiments, the solution comprises from 0.01% to 1% acid, such as 0.02-0.5%, 0.025-0.25%, or 0.05-0.15%. In some examples, the solution includes 0.1% hydrochloric acid.

Extraction may be performed at a temperature ranging from 0-100° C. In some embodiments, extraction is performed at a temperature ranging from 20-70° C., or at ambient temperature. Extraction may be performed for a duration ranging from several minutes to several days. To increase extraction efficiency, the plant material and solution may be mixed or agitated during extraction, such as by grinding the plant material during extraction, stirring the mixture, shaking the mixture, or homogenizing the mixture. In some embodiments, the extraction may be repeated one or more times with fresh solution to increase recovery of polyphenols and other non-polyphenolic compounds from the plant material. The liquid phases from each extraction cycle are then combined for further processing.

The liquid phase can recovered, and the residual solids, or pulp, are discarded. Recovering the liquid phase may comprise decanting the liquid from the remaining solids and/or filtering the liquid phase to remove residual solids. The solvent (alcohol, ester, or combination thereof) can be removed from the liquid solution by any suitable means, such as evaporation (e.g., roto-evaporation), to produce an aqueous extract containing the biologically-active components in a mildly acidic solution.

In certain embodiments where the plant material includes a significant amount of oils, or lipids, an initial extraction of nonpolar components may be performed before extracting the polyphenols and other polar, non-polyphenolic compounds. Nonpolar components may be extracted by homogenizing the plant material in a nonpolar solvent, e.g., hexanes, heptanes, or a combination thereof. The solvent layer including the extracted nonpolar components is separated from the plant material and discarded.

The aqueous plant extract may be further purified by suitable means, e.g., extraction, chromatographic methods, distillation, etc., to remove non-polyphenolic compounds and/or to increase the concentration of polyphenols relative to other compounds in the extract.

The aqueous plant extract may be dried, for example by freeze-drying or other low-temperature drying methods, and ground to a powder to provide a dried plant extract. In some embodiments, the dried plant extract comprises 0.01 wt % to 25 wt % total polyphenols, such as 0.01 wt % to 10 wt %, 0.01 wt % to 5 wt %, 0.01 wt % to 2.5 wt %, 0.01 wt % to 1 wt %, 0.01 wt % to 0.5 wt %, 0.02 to 0.25 wt %, or 0.03-0.1 wt % total polyphenols. In certain embodiments, the dried plant extract further comprises non-polyphenolic compounds. For example, the dried plant extract may comprise 0.01-1 mg/g gallic acid, such as 0.05-0.5 mg/g or 0.09-0.25 mg/g gallic acid, and/or 0.001-0.1 mg/g trans-caftaric acid, such as 0.005-0.05 mg/g or 0.01-0.025 mg/g trans-caftaric acid.

The aqueous plant extract may be concentrated to a smaller volume, e.g., by evaporation, and used as an aqueous plant extract. In other embodiments, the aqueous plant extract is mixed with a carrier before drying and grinding. Suitable carriers include, for example, diatomaceous earth, silica, maltodextrin, ground grain (e.g., corn), meals (e.g., soybean or cottonseed meal) by-products (e.g., distiller's dried grains, rice hulls, wheat mill run), clays (e.g., bentonite), and combination thereof. The plant extract may be combined with a carrier in a ratio ranging from 10:1 to 1:10 by weight, such as from 5:1 to 1:5. For example, the plant extract may be mixed with diatomaceous earth in a ratio of 3:1 by weight.

In some embodiments, the composition and/or combinations disclosed herein can be formulated in any suitable form, including a powder, a granule, a pellet, a solution, or a suspension. Certain disclosed composition and/or combination embodiments can be formulated as a dry, free-flowing powder, which can be used for direct inclusion into a commercially-available feed, food product, or as a supplement to a total mixed ration or diet. In some embodiments, the powder can be mixed with either solid or liquid feed, or with water. In another embodiment, the compositions and/or combinations disclosed herein can be formed into pellets.

In some embodiments, the compositions and/or combinations disclosed herein can have an average particle size selected to be compatible with a feedstuff or companion animal food or other components with which the compositions and/or combinations may be admixed. The term “compatible” as used herein means that the particle size is sufficiently similar to reduce or eliminate particle size segregation when the composition or combination is admixed with the feedstuff or companion animal food or other components. For example, if a combination embodiment is admixed with a feedstuff or companion animal food or component having an average particle size of 50-200 μm, the combination (or the compositions included therein) may have a similar average particle size, e.g., from 80-120% of the food or feedstuff/component particle size with which the combination is admixed.

In some embodiments, the amount of each of the yucca, quillaja, direct-fed microbial, vitamin D compound(s), chromium compound(s), and plant extract(s) can range from 5 ppm to 2,000 ppm based on the total dry weight basis of a feedstock, companion animal food, or the combination, such as 5 ppm to 1,000 ppm, 5 ppm to 500 ppm, 5 ppm to 250 ppm, or 5 ppm to 100 ppm. In some embodiments, the amount of each of the yucca, quillaja, direct-fed microbial, vitamin D compound(s), chromium compound(s), and plant extract(s) can range from 5 ppm to 10 ppm based on the total dry weight basis of a feedstock, companion animal food, or the combination.

In some embodiments, a primary composition and any one or more of yucca, quillaja, a direct-fed microbial, a vitamin D compound(s), a chromium compound(s), or a plant extract(s) can be administered in a ratio from 1,000:1 to 1:1,000 by weight. In some embodiments, the components of the combination can be combined with the feedstock or companion animal food individually or together as a single combination. In some embodiments, the components of the combination can be combined with the feedstock or companion animal food just prior to administration to the animal. For example, the combination can be mixed into a feedstuff or companion animal food in an amount ranging from 0.1 to 20 kg per ton of feed, or in an amount ranging from 0.01% to 2.5% by weight.

In some embodiments, components may be incorporated in different manners, including as a composition and/or as a combination. For example, the composition and/or combination may comprise a component 1 selected from: 1A) glucan (e.g., β-glucan); 1B) silica; 1C) mineral clay; 1D) mannan; 1E) plant extract; 1F) glucan (e.g., β-glucan) and silica; 1G) glucan (e.g., β-glucan) and mineral clay; 1H) glucan (e.g., β-glucan) and mannan; 1I) glucan (e.g., β-glucan) and plant extract; 1J) silica and mineral clay; 1K) silica and mannan; 1L) silica and plant extract; 1M) mineral clay and mannan; 1N) mineral clay and plant extract; 1O) mannan and plant extract; 1P) glucan (e.g., β-glucan), silica and mineral clay; 1Q) glucan (e.g., β-glucan), silica and mannan; 1R) glucan (e.g., β-glucan), silica and plant extract; 1S) glucan (e.g., β-glucan), mineral clay and mannan; 1T) glucan (e.g., β-glucan), mineral clay and plant extract; 1U) glucan (e.g., β-glucan), mannan and plant extract; 1V) silica, mineral clay and mannan; 1W) silica, mineral clay and plant extract; 1X) mineral clay, mannan and plant extract; 1Y) glucan (e.g., β-glucan), silica, mineral clay and mannan; 1Z) glucan (e.g., β-glucan), silica, mineral clay and plant extract; 1AA) silica, mineral clay, mannan and plant extract; 1AB) glucan (e.g., β-glucan), silica, mineral clay, mannan and plant extract; 1AC) quillaja; 1AD) yucca; 1AE) a vitamin D species; 1AF) quillaja and yucca; 1AG) quillaja and a probiotic; 1AH) yucca and a vitamin D species; 1AI) quillaja, yucca and a vitamin D species; 1AJ) Yucca schidigera; 1AK) Quillaja saponaria; 1AL) Bacillus coagulans; 1AM) Yucca schidigera and Bacillus coagulans; 1AN) Quillaja saponaria and Bacillus coagulans; 1AO) Yucca schidigera, and Quillaja saponaria; 1AP) Yucca schidigera, Quillaja saponaria and Bacillus coagulans; 1AQ) a direct-fed microbial; 1AQ1) endoglucanohydrolase; 1AQ2) an antimicrobial; 1AR) an antibiotic; 1AS) Virginamycin; 1AT) an anticoccidial agent, for example Salinomycin; 1AU) an antifungal; 1AV) an antiviral; 1AW) an antiparasitic; 1AX) a vaccine; 1AY) a coating agent; 1AZ) a chromium compound.

The composition and/or combination may also comprise a component 2. With respect to the component 1 embodiments, the component 2 may be, in a combination with 1A to 1AZ: 2A) quillaja; 2B) yucca; 2C) a vitamin D species; 2D) quillaja and yucca; 2E) quillaja and a vitamin D species; 2F) yucca and a vitamin D species; 2G) quillaja, yucca and a vitamin D species; 2H) Yucca schidigera; 2I) Quillaja saponaria; 2J) Bacillus coagulans; 2K) Yucca schidigera and Bacillus coagulans; 2L) Quillaja saponaria and Bacillus coagulans; 2M) Yucca schidigera, and Quillaja saponaria; 2N) Yucca schidigera, Quillaja saponaria and Bacillus coagulans; 2O) a direct-fed microbial; 2OA) endoglucanohydrolase; 2OB) an antimicrobial; 2P) an antibiotic; 2Q) Virginamycin; 2R) an anticoccidial agent, for example Salinomycin; 2S) an antifungal; 2T) an antiviral; 2U) an antiparasitic; 2V) a vaccine; 2W) a coating agent; or 2X) a chromium compound. A person of ordinary skill in the art will understand that any of 2A to 2X may be combined with any of 1A to 1AZ, to form any and all compositions and/or combinations between such substituents.

The composition and/or combination may comprise a component 3. With respect to the component 1 embodiments 1A to 1AZ and the component 2 embodiments 2A to 2X, component 3 may be, in combination with 1A to 1AZ and 2A to 2X: 3A) an antimicrobial; 3B) an antibiotic; 3C) Virginamycin; 3D) an anticoccidial agent, for example Salinomycin; 3E) an antifungal; 3F) an antiviral; 3G) an antiparasitic; 3H) a vaccine; 31) a coating agent; 3J) a direct-fed microbial; 3K) endoglucanohydrolase; or 3L) a chromium compound.

A person of ordinary skill in the art will understand that any of 3A to 3L may be combined with any of 1A to 1AZ and any of 2A to 2X, to form any and all compositions and/or combinations between such substituents.

The composition and/or combination may further comprise a component 4. With respect to the component 1 embodiments 1A to 1AZ the component 2 embodiments 2A to 2X, and the component 3 embodiments 3A to 3L, component 4 may be, in combination with 1A to 1AZ, 2A to 2X, and 3A to 3L: 4A) an antimicrobial; 4B) an antibiotic; 4C) Virginamycin; 4D) an anticoccidial agent, for example Salinomycin; 4E) an antifungal; 4F) an antiviral; 4G) an antiparasitic; 4H) a coating agent; 41) a direct-fed microbial; 4J) endoglucanohydrolase; or 4K) a chromium compound.

A person of ordinary skill in the art will understand that any of 4A to 4K may be combined with any of 1A to 1AZ, any of 2A to 2X, and any of 3A to 3L, to form any and all compositions and/or combinations between such substituents.

The composition and/or combination may further comprise a component 5. With respect to the component 1 embodiments 1A to 1AZ, the component 2 embodiments 2A to 2X, the component 3 embodiments 3A to 3L, and the component 4 embodiments 4A to 4K, component 5 may be, in combination with 1A to 1AZ, 2A to 2X, 3A to 3L, and 4A to 4K: 5A) an antimicrobial; 5B) an antibiotic; 5C) Virginamycin; 5D) an anticoccidial agent, for example Salinomycin; 5E) an antifungal; 5F) an antiviral; 5G) an antiparasitic; or 5H) a direct-fed microbial; 5I) endoglucanohydrolase; or 5J) a chromium compound.

A person of ordinary skill in the art will understand that any of 5A to 5J may be combined with any of 1A to 1AZ, any of 2A to 2X, any of 3A to 3L, and any of 4A to 4K to form any and all compositions and/or combinations between such substituents.

The composition and/or components of the combination may be formulated in any suitable form, including a powder, a granule, a pellet, a solution, or a suspension. In one embodiment, the composition and/or components of the combination are dry, free-flowing powder(s) suitable for direct inclusion into a commercially-available feed, food product or as a supplement to a total mixed ration or diet. The powder may be mixed with either solid or liquid feed or with water. In another embodiment, the composition and/or components of the combination can be formed into pellets.

In some embodiments the composition and/or combination may be a powder top coated onto a feedstuff using a coating agent. In some embodiments the feed is mixed with coating agent in a mixer. The composition is added to feedstuff and mixed until all components are suitably blended.

In some embodiments, the combination and/or composition was admixed with a feedstuff. In certain embodiments the combination and/or composition is formulated to be suitable to form a homogeneous mixture with the feedstuff, such as by crushing, crumbling, grinding or otherwise sizing the combination. Alternatively, the combination and/or composition may be formulated as a solution, suspension or slurry. In embodiments where the combination comprises two or more components, the components may be formulated separately or substantially together. The components may also be admixed with the feedstuff sequentially, in any order, or substantially simultaneously.

### IV. Methods of Use

Embodiments of the compositions disclosed herein can be used for feeding animals and can provide additional nutritional benefit to the animals to help support and/or maintain the animals' overall health and well-being, such as to help increase longevity of the animal, help boost immunity to disease, and other health benefits. In some embodiments, the methods can comprise, consist essentially of, or consist of combining a primary composition comprising, consisting essentially of, or consisting of a β-glucan (e.g., β-1,3 (4)glucan), silica, mineral clay, mannans, β-1,3 (4)-endoglucanohydrolase, and any combination thereof with any one or more of yucca, quillaja, a direct-fed microbial, a vitamin D species, a chromium compound, or a plant extract. The method can further comprise administering the combination to an animal alone or in combination with a feedstuff or companion animal food. In some embodiments, the methods concern administering a combination of compositions, or compositional components disclosed herein in combination with a companion pet food. In exemplary embodiments, the companion pet food can be a wet or dry canine food or a wet or dry feline food. In some embodiments, the canine food and/or the feline food can be a normal or specialized diet food.

In some embodiments, the combinations can be used to help promote health in an animal at risk of developing a disease due to old age or immune deficiencies. In such embodiments, the animal can be affirmatively selected based on one or more factors that can contribute to its susceptibility to a particular disease. Such factors can include the animal's age, decreased immunity, exposure to stressors (e.g., heat stress, crowding, work load, chemotherapy, anti-inflammatory therapy), gastrointestinal disturbances (e.g., diarrheal diseases), or combinations thereof. In exemplary embodiments, the animal can be a companion animal, particularly a canine or feline, that is susceptible to disease due to age (e.g., young or geriatric animals) or decreased immune function. In other embodiments, the method can be used to ameliorate signs or symptoms of disease in an animal that is suffering or afflicted with the disease. Exemplary embodiments can comprise administering the combination to an animal to help ameliorate signs or symptoms of any one or more of the following: stiffness, impaired cognitive function and/or memory (including age-related memory impairment and/or cognitive degradation), dermatitis (e.g., atopy, flea-allergy dermatitis, food allergy dermatitis, contact dermatitis, and juvenile dermatitis), osteoarthritis, conjunctivitis/uveitis, pyometra, prostatitis, orchitis cystitis, renal disease, autoimmune disorders (e.g., Pemphigus vulgaris/foliaceus, lupus, autoimmune hemolytic anemia, and rheumatoid arthritis), endocrine disorders (e.g., Addison's Disease, Cushing's disease, hyper and hypothyroidism, diabetes mellitus, and diabetes insipidus), inflammatory bowel disease, hemorrhagic gastroenteritis, otitis, viral diseases (Distemper virus, Parvovirus, coronavirus, herpesvirus, influenza virus, hepatitis virus), bacterial diseases (Salmonellosis, Bordetella bronchiseptica, Lyme's disease, leptospirosis, Brucellosis), fungal diseases (Blastomycosis, Aspergillosis, Cryptococcus, Coccidioidomycosis, Microsporum/Trichophyton, Histoplasmosis), parasitic diseases (coccidiosis, giardiasis, hookworms, roundworms, whipworms, tapeworms, mange), meningitis-arteritis/Encelphalitis, acquired myasthenia gravis, neoplasia (lymphosarcoma, osteosarcoma, hemangiosarcoma, mast cell tumor, squamous cell carcinoma, melanoma, leukemia, mammary tumors, lung cancer, testicular cancer, transitional cell carcinoma, brain tumor), laminitis, founder, heart disease, epilepsy, hepatitis, pancreatitis, gingivitis, and combinations thereof.

Animals disclosed herein can exhibit a response, or a combination of responses, to the combinations (or a component thereof) disclosed herein. In some embodiments, these responses can be detected and measured to determine whether an animal's health is supported by administration of the combination to the animal. In particular disclosed embodiments, one or more factors (or endpoints) can be used to determine an animal's response to the combination. In some embodiments, a factor that can be examined is the ability of the combination (or a component thereof) to act as a surfactant to, for example, reduce the surface tension between the material in a biological lumen of the animal and the epithelial cell layer, to coat epithelial cells, to deliver digestive enzymes that can modify nutrient transport, and to bind pathogenic bacteria. In another embodiment, a factor that can be examined is the ability of the combination (or a component thereof) to act as an emulsifier by dispersing molecules thereby facilitating nutrient transport and/or increase the exposure of antigens to M cells and other antigen-sensing cells in an animal's gut. In yet other embodiments, the anti-oxidant, anti-inflammatory, anti-microbial, and/or anti-hypertensive properties of the combination (or a component thereof) can be factors that are examined. In some embodiments, the ability of the combination to beneficially affect immune modulation, metabolic regulation, nutrient utilization and/or transport, endocrine and neuroendocrine regulation, and longevity (or lifespan) can be determined.

In some embodiments, the combinations can be used to help increase longevity of an animal and therefore help prolong its lifespan for a longer period of time (e.g., days, weeks, years) than an animal not administered the combination. In such embodiments, the combination can be administered to the animal at any point during the animal's lifespan. In some embodiments, the combination can be administered to an animal that has reached an age ranging from an age that is less than 50% of its average lifespan to an age that is 90% (or higher) of its average lifespan. “Average lifespan,” as used herein, is understood to be the average lifespan of an animal species or breed (or mix of breeds) as understood by a person of ordinary skill in the veterinary arts. For example, some animals can be administered the combination when it has reached an age ranging from 10% of its average lifespan to 80% of its average lifespan, or when it has reached an age ranging from 25% of its average lifespan to 75% of its average lifespan.

The age of the animal that is to be administered the combination can vary with the species or breed (or mixed breed) of the animal. For example, some animal species, breeds, or mixed breeds may have average lifespans that are longer or shorter than another species of animal; therefore, the point at which the combination is administered can be guided by the type of animal to which the combination is being administered. Solely by way of example, the combination can be administered to companion animals like canines and felines that are young companion animals, which (with respect to canines and felines) is understood herein to mean an age ranging from birth to the age of two years. Other animal species disclosed herein, however, may still be considered young companion animals but can have an upper age limit higher than two years (e.g., 10% to 200% or higher) or an upper age limit lower than two years (e.g., 10% to 90% or lower). In yet another example, the combination can be administered to a geriatric canine or feline, which (with respect to canines and felines) is understood herein to mean an age ranging from six years to the animal's age at the time of death. Other animal species disclosed herein, however, may still be considered geriatric companion animals, but can have a lower age limit higher than six years (e.g., 10% to 200% or higher) or a lower age limit lower than six years (e.g., 10% to 50% or higher). In particular disclosed embodiments, the combination can be administered to the animal after birth (e.g., days, weeks, or months after birth), and administered once or more daily over a period of days, weeks, and even years. In other disclosed embodiments, the combination can be administered to the animal once it has reached an age of, for example, three years to ten years, such as six years to ten years, or six years to eight years.

In some embodiments, the combination can be administered daily to the animal at time intervals believed or determined to be effective for achieving a beneficial result. The combinations can be administered in a single dose daily or in divided doses throughout the day. In some instances, one or more individual compositions disclosed herein (or any compositional components thereof) may be administered to the animal at a first time, and remaining compositions (or compositional components thereof) may be administered individually or in combination at one or more subsequent times during the same day.

Combination embodiments disclosed herein, when administered to an animal, may produce a concomitant change in a level of, for example, an immune system biomarker or an inflammation biomarker in the animal by at least 5%, at least 10%, at least 20%, at least 30%, at least 50%, at least 75%, at least 100%, at least 200%, or at least 500%, such as from 5-600%, from 10-500%, from 10-200%, or from 10-100%, compared to an average level of the biomarker in an animal that has not received the combination. The change may be an increase or a decrease, depending on the particular biomarker.

In some embodiments, a biomarker level is assessed by measuring a concentration of messenger RNA (mRNA) encoding the biomarker. In some embodiments, when administered to an animal, the combinations may produce a concomitant change in a concentration of mRNA encoding an immune system biomarker or an inflammation biomarker in the animal by at least 5%, at least 10%, at least 20%, at least 30%, at least 50%, at least 75%, at least 100%, or at least 200%, or at least 500%, such as from 5-600%, from 10-500%, from 10-200%, or from 10-100% compared to an average concentration of the mRNA in an animal that has not received the combination. The change may be an increase or a decrease, depending on the particular mRNA.

In some embodiments, administration of the composition and/or combination may produce a concomitant change in a level of innate defense mechanisms of fish prior to exposure to a pathogen, or improve survival following exposure to a specific pathogen. Markers of improved innate immune response may include: total leucocyte count; respiratory burst (release of superoxide anion); phagocytic index and activity; and lysozyme activity.

Abnormal changes in total and differential blood cell counts in fish, such as anaemia, leukopaenia, leukocytosis and thrombocytopaenia, may result from diseases, but may also indicate stress, toxic exposure, hypoxia and changes in reproductive status.

Due to the nucleated nature of red blood cells (erythrocytes) in fish, white blood cells (leukocytes), which serve as an indicator of health, cannot be distinguished using automated cell counting procedures without lysis of erythrocytes and are usually manually counted using a haemocytometer. Differential leukocyte and haemocyte enumerations, which also serve as health indicators, are generally performed either on stained smears or with a haemocytometer in fish and crustacea, respectively. The disadvantage of manual enumeration is the statistical limitation associated with counting between 100 to 200 cells, the typical range in differential leukocte procedures.

Flow cytometry is an instrumental technique in which a stream of suspended particles is interrogated by one or more lasers. Particles are analyzed and differentiated on the basis of their light-scattering properties, auto- or labelled fluorescence, or a combination of both.

The major advantages of flow cytometry technology are the ability to differentiate and enumerate several thousands of particles per second, and to physically sort multiple populations simultaneously into collection vessels. In haematological applications, the capability to obtain accurate and precise total and 5 differential blood counts on so many more cells than practically achievable with manual methods, in a fraction of the time, is thus dependent only on the ability to accurately discriminate between cell types.

Several reactive oxygen species (ROS) are produced by fish phagocytes during the respiratory burst. Once bacteria or fungi are engulfed by leucocytes, the host's NADPH-oxidase is activated, which in turn increases oxygen consumption and subsequently produces ROS such as superoxide anion (O₂⁻), hydrogen peroxide (H₂O₂), hydroxyl radical (OH) and singlet oxygen (¹O₂). The release of superoxide anion is known as the respiratory burst, and the ROS released and/or formed may be are bactericidal.

Phagocytosis is an essential component of the non-specific immune response against infectious agents in teleosts. This process involves the recognition and attachment of foreign particles, including pathogens, engulfment and digestion by the phagocyte. In vitro assays have been used for studying fish macrophage phagocytic activity, thereby providing an avenue for evaluating immunocompetence in fish. In vitro assays have also provided insight for non-specifically enhancing disease resistance in finfish aquaculture, and have served as immunological biomarker tests to assess aquatic environmental health.

Lysozyme found in cutaneous mucus, peripheral blood and certain tissues rich in leucocytes, is an enzyme which catalyzes the hydrolysis of N-acetyl muramic acid and N-acetyl glucosamine of peptidoglycan in bacterial cell walls. This protein plays a crucial role in the defense system.

In other embodiments, administration of the composition and/or combination may produce a concomitant change in a level of innate defense mechanisms of crustaceans prior to exposure to a pathogen, or improve survival following exposure to a specific pathogen. Markers of improved innate immune response in crustaceans may include: total hemocyte count; phagocytic activity; phenoloxidase (PO) and prophenoloxidase (ProPO) activity; antibacterial activity; plasma protein concentration

Haemocytes play a central role in crustacean immune defense. Firstly, they remove foreign particles in the hemocoel by phagocytosis, encapsulation and nodular aggregation. Secondly, haemocytes take part in wound healing by cellular clumping and initiation of coagulation processes through the release of factors required for plasma gelation.

The hemogram consists of the total haemocyte count (THC) and the differential haemocyte count (DHC). For the DHC, most researchers agree with the identification of three cell types in penaeid shrimp: large granule haemocytes (LGH), small granule haemocytes (SGH) and agranular haemocytes or hyaline cells (HC).

THC can be easily determined using a hemocytometer, whereas determination of DHC requires a more complex haemocyte identification. DHC can be determined by using morphological criteria such as size and shape of cells and the difference of haemocyte refractivity using a phase contrast microscope. Although this technique is rapid, it should be mentioned that when using this technique it is easy to obtain large variations in results possibly due to interpretation errors.

Different haemocyte types can be determined using cytochemical studies of enzyme activity detection or specific stains. The results obtained from cytochemical stains for penaeid shrimp indicate that these specific stainings can differentiate between the types of haemocytes and provide additional information on their functions. An alternative method for cell identification is the use of monoclonal antibodies (mAbs) in order to find antigenic markers of different cell types. Using mAbs against different subpopulations of haemocytes separated by isopycnic centrifugation on a Percoll gradient, it has been found in P. japonicus that HC share epitopes with SGH, and that an antigen was specifically expressed for LGH. Monoclonal antibodies could be considered as powerful tools for the development of haemocyte lineages and haemocyte proliferation studies, as well as for the isolation and study of plasma components.

Phagocytosis is the most common reaction of cellular defense. During phagocytosis, particles or microorganisms are internalized into the cell which later forms a digestive vacuole called the phagosome. The elimination of phagocyted particles involves the release of degradative enzymes into the phagosome and the generation of reactive oxygen intermediates (ROIs). This last process is known as the respiratory burst. The first ROI generated during this process is the superoxide anion. Subsequent reactions will produce other ROIs, such as hydrogen peroxide, hydroxyl radicals and singlet oxygen. Hydrogen peroxide can be converted to hypochlorous acid via the myeloperoxidase system, forming a potent antibacterial system.

Despite the limited number of studies focusing on respiratory burst in penaeid shrimp, the actual results are very interesting in view of their value as biomarker of environmental disturbances. Furthermore, the importance of respiratory burst as a microbicidal mechanism in penaeid shrimp is strongly suggested by the fact that pathogenic bacteria of shrimp have developed ways of circumventing this mechanism. In P. Íannamei, O₂ generation is not produced when virulent Vibrio Íulnificus is used as elicitor, as opposed to strong stimulation generated by V. Iginolyticus and other bacteria, such as Escherichia coli.

The PO is responsible for the melanization process in arthropods. The PO enzyme results from the activation of the ProPO enzyme. The ProPO activating system has been very well studied in crustaceans. Using these different approaches, the function of the ProPO system can be better understood in relation to the health status of shrimp. Some studies have shown that ProPO could be used as health and environmental markers because changes are correlated with infectious state and environmental variations, this issue which has recently been confirmed also at the gene expression level. Phenoloxidase, which has been detected in a wide range of invertebrates, is activated by several microbial polysaccharides, including β-1,3-glucan from fungal cell walls and peptidoglycans or lipopolysaccharides from bacterial cell walls.

Antibacterial peptides and proteins have been well studied in arthropods, mainly in insects and chelicerata, where the families of antimicrobial molecules have been isolated and characterized. In crustacean, some studies have shown the ability of crustacean haemolymph to inhibit bacterial growth. Several antibacterial proteins, active in vitro against Gram-positive and Gram-negative bacteria, were found in C. maenas.

In the literature there are reports showing that antibacterial activity in crustaceans can be considered as an environmental marker. Therefore, many researchers have developed quantitative antibacterial assays based on inhibition of bacterial growth on agar plate (zone inhibition assay and colony-forming units (CFU) inhibition assay), or in liquid medium on microtiter plates (turbidometric assay), to detect the antibacterial ability in crustacean haemolymph. Using the CFU inhibition technique, antibacterial activity has been found in granular haemocytes of the shore crab C. maenas and in other crustacean species. It has been reported that a potent antibacterial activity in the serum of C. sapidus, using the zone inhibition assay and turbidimetric test. Using the CFU inhibition assay, bactericidal activity against Gram negative bacteria have been described in the haemolymph of P. monodon. In P. Íannamei, strong antibacterial activity of plasma against different marine bacteria has been observed, using a turbidimetric assay.

In recent years blood metabolites have been investigated as a tool for monitoring physiological condition in wild or cultured crustaceans exposed to different environmental conditions. Hemocyanin is the major hemolymph constituent (>60%); the remaining proteins (in order of concentration) include coagulogen, apohemocyanin, hormones, and lipoproteins. Blood protein levels fluctuate with changes in environmental and physiological conditions and play fundamental roles in the physiology of crustaceans from O₂ transport to reproduction up to stress responses. In fact, moulting, reproduction, nutritional state, infection, hypoxia, and salinity variations are the major factors affecting the relative proportions and total quantities of the hemolymph proteins.

The shrimp immune system response is largely based on proteins. These are involved for example in recognizing foreign particles and in trapping foreign invading organisms and prevent blood loss upon wounding. Recently, it has been shown that shrimp are well adapted to use protein as a source of energy and molecules. Blood protein concentration has been found to be related to nutritional condition in a number of crustaceans. The concentration of protein in the blood is a possible index of nutritional condition, which decreases in starved prawns and lobsters. The moult cycle imposes constraints on protein levels, blood-proteins typically drop just before moulting as water is taken up and protein is used to synthesize the new exoskeleton. Protein levels then gradually build up again after ecdysis as water is replaced by tissue. Consequently, measuring the blood protein concentration of a crustacean sample group can provide valuable information to identify its condition. The concentration of protein in the blood is directly proportional to the refractive index of the blood. Measurements of the blood refractive index therefore offer potential as a field method for assessing the nutritional condition of prawns.

Colorimetric procedures are generally the preferred choice to measure serum protein concentration; however, they are expensive, time consuming, and not easily performed in the field. Because of ease, rapid mode of operation, and small amount of material required, measuring serum protein concentration using a refractometer provided a nondestructive field method to assess crustacean's physiological state (stress, immunoresponse, nutrition status, molt, etc.) without any need of laboratory facilities; the refractometer is a simple, small portable instrument that can be used in the field or on crustacean farms.

When administered to an animal, embodiments of the disclosed combination exhibit measurable effects on at least some biomarkers associated with inflammation. Exemplary biomarkers include, but are not limited to, tumor necrosis factor alpha (TNF-α), nuclear factor kappa B (NF-κB), interferon gamma (IFN-γ), interleukin-6 (IL6), interleukin-8 (IL8), interleukin-1β (IL-1β), cellular COX-2 gene expression, L-selectin, interleukin-8 receptor (IL8R), cellular lipoxygenase (LOX), C-reactive protein (CRP), macrophage inflammatory protein 1-alpha (MIP) gene expression, eicosanoids (localized cellular products produced by COX and LOX), alpha-1-antitrypsin (AAT), haptoglobin, fibrinogen, uric acid/urate, homocysteine, adiponectin, and/or toll-like receptors (TLRs). Administering an embodiment of the disclosed combination to an animal may promote a reduction in an in vivo level of at least one biomarker, thereby helping to maintain the animal's overall health by helping to reduce the prevalence and/or severity of symptoms or signs associated with an inflammatory disorder characterized by an elevation in one or more of these biomarkers including, but not limited to, arthritis, hoof disorders, dermatitis, eczema, atherosclerosis, cystitis, inflammatory bowel disease, hemorrhagic gastroenteritis, meningitis-arteritis, encephalitis, acquired myasthenia gravis, hepatitis, pancreatitis, gingivitis, degenerative neurological conditions, diabetes, and obesity.

Without wishing to be bound by any particular theory of operation, the compositions and combinations disclosed herein can enhance the animal's immune system (e.g., the innate and/or adaptive immune system). For example, some embodiments of the compositions and/or combinations disclosed herein can affect or change (e.g., increase or reduce by 10% to 90%, such as 10%, 20%, 30%, 40%, 50%, 60%, 70%, 80%, or 90%) levels of immune biomarkers including, but not limited to, neutrophil L-selectin, IL-1β and/or gene expression of C-reactive protein (gene name “CRP”), mannose binding lectin 2 (gene name “Mbl2”), amyloid P component, serum (gene name “Apcs”), Interleukin 5 (gene name “Il5”), Interferon alpha 1 (gene name “Ifna1”), chemokine ligand 12 (gene name “Ccl12”), granulocyte macrophage colony stimulating factor (gene name “Csf2”), interleukin 13 (gene name “Il13”), interleukin 10 (gene name “Il10”), trans-acting T-cell-specific transcription factor-3 (gene name “Gata3”), signal transducer and activator of transcription 3 (gene name “Stat3”), C3 convertase (gene name “C3”), toll-like receptor 3 (gene name “Tlr3”), chemokine ligand 5 (gene name “Ccl5”), interferon-induced GTP-binding protein (gene name “Mx2”), NF kappa B-1 (gene name “Nfkb1”), NF kappa B1-alpha (gene name “NFKBIA”), toll-like receptor 9 (gene name “Tlr9”), C—X—C motif chemokine 10 (gene name “Cxcl10”), cluster of differentiation 4 (gene name “Cd4”), interleukin 16 (gene name “Il6”), chemokine ligand 3 (gene name “Ccl3”), C-C chemokine receptor Type 6 (gene name “Ccr6”), cluster of differentiation 40 (gene name “Cd40”), RIG-1-like receptor double stranded RNA helicase (gene name “Ddx58”), interleukin 18 (gene name “Il18”), c-Jun protein (gene name “Jun”), tumor necrosis factor (gene name “Tnf”), tumor necrosis factor receptor associated factor 6 (gene name “Traf6”), signal transducer and activator of transcription 1 (gene name “Stat1”), interferon beta-1 (gene name “Ifnb1”), cluster of differentiation 80 (gene name “Cd80”), toll-like receptor 1 (gene name “Tlr1”), toll-like receptor 6 (gene name “Tlr6”), MAP kinase 8 (gene name “Mapk8”), nucleotide-binding oligomerization domain-containing protein 2 (gene name “Nod2”), C-C chemokine receptor Type 8 (gene name “Ccr8”), interleukin-1 receptor-associated kinase 1 (gene name “Irak1”), CD1 family of glycolipid antigen-presenting MHC-like molecules (gene name “Cd1d1”), signal transducer and activator of transcription 4 (gene name “Stat4”), IAA-amino acid hydrolase (gene name “11r1”), Fas ligand (TNF superfamily, member 6) (gene name “Fas1g”), interferon regulatory factor 3 (gene name “Irf3”), phosphorylated interferon-alpha receptor 1 subunit (gene name “Ifnar1”), Nramp1 (gene name “Slc11a1”), toll-like receptor 4 (gene name “Tlr4”), cluster of differentiation 86 (gene name “Cd86”), caspase 1 (gene name “Casp1”), C-C chemokine receptor Type 5 (gene name “Ccr5”), intercellular adhesion molecule 1 (gene name “Icam1”), cathelcidin antimicrobial protein (gene name “Camp”), toll-like receptor 7 (gene name “Tlr7”), interferon regulatory factor 7 (gene name “Irf7”), RAR-related orphan receptor C (gene name “Rorc”), cluster of differentiation 40lg (gene name “Cd40lg”), T-box transcription factor 21 (gene name “Tbx21”), caspase 8 (gene name “Casp8”), interleukin 23a (gene name “Il23a”), cluster of differentiation 14 (gene name “Cd14”), cluster of differentiation 8a (gene name “Cd8a”), chemokine receptor 3 (gene name “Cxcr3”), forkhead box P3 (gene name “Foxp3”), lipopolysaccharide-binding protein (gene name “Lbp”), MAP kinase 1 (gene name “Mapk1”), myeloid differentiation primary response gene 88 (gene name “Myd88”), signal transducer and activator of transcription 6 (gene name “Stat6”), Agrin and/or interleukin 33 (gene name “IL33”). As disclosed in U.S. Pat. No. 8,142,798, which is incorporated herein by reference, some embodiments of the combination also can augment an animal's adaptive immune system, e.g., by increasing response to a vaccine. In some embodiments, antibody levels, such as IgG levels, may be increased, relative to an animal that has received a vaccine but has not been administered the combinations disclosed herein. Some embodiments of the combination can affect levels of inflammation biomarkers, e.g., COX-2, IL-1(3, tumor necrosis factor alpha (TNF-α), interleukin-8 receptor (IL8R), and/or L-selectin. In some embodiments, animal immune cells can be increased by 10% to 90%, such as 10% to 80%, 20% to 70%, or 30% to 60%.

In one embodiment, when incorporated directly into a feedstuff, the combination can be added in amounts ranging from 0.1 to 20 kg per ton (2000 pounds) of feed. In some embodiments, combination embodiments can be added to animal feedstuff in amounts from 0.5 kg to 10 kg per ton of feed. In certain embodiments, combination embodiments can be added to feedstuff in amounts ranging from 1 to 5 kg per ton of feed.

For some embodiments, such as with livestock cattle, the combination can be provided in the range of from 10 grams per head per day to 70 grams per head per day, such as from 45 grams per head per day to 70 grams per head per day, or from 50 grams per head per day to 60 grams per head per day. A person of ordinary skill in the art will appreciate that the amount of the combination fed can vary depending upon a number of factors, including the animal species, size of the animal and type of the feedstuff to which the combination is added. In some embodiments, individual components of the combination, such as yucca, quillaja, a direct-fed microbial, a vitamin D species, or a plant extract, may be present in the amounts discussed above; therefore, the amount of the portion of the combinations comprising all 5 components can range from 25 ppm to 10,000 ppm based on the total dry weight basis of a feedstuff, companion animal food, or the combination, such as 25 ppm to 5,000 ppm, or 25 ppm to 2,500 ppm, or 25 ppm to 1,250 ppm, or 25 ppm to 500 ppm. Similarly, in embodiments comprising four such components, the amount of the portion of the combination comprising these four components can range from 20 ppm to 8,000 ppm, such as 20 ppm to 4,000 ppm, or 25 ppm to 2,000 ppm, or 25 ppm to 1,000 ppm, or 25 ppm to 400 ppm. Combination embodiments comprising three such components can include an amount of the portion of the combination comprising the three components that can range from 20 ppm to 6,000 ppm, such as 20 ppm to 3,000 ppm, or 25 ppm to 1,500 ppm, or 25 ppm to 750 ppm, or 25 ppm to 300 ppm. Combination embodiments comprising two such components can include an amount of the portion of the combination comprising the two components that can range from 20 ppm to 4,000 ppm, such as 20 ppm to 2,000 ppm, or 25 ppm to 1,000 ppm, or 25 ppm to 500 ppm, or 25 ppm to 200 ppm. Alternatively, the combination can be fed directly to animals as a supplement in amounts of from 0.01 gram to 20 gram per kilogram of live body weight per day, such as from 0.01 gram to 10 gram per kilogram, 0.01 gram to 5 gram, 0.01 gram to 1 gram, or 0.015 gram to 1 gram.

In some embodiments, the disclosed composition and/or combination can be administered to aquatic animals to obtain one or more beneficial results. For example, embodiments of the composition and/or combination may be used to prevent and/or treat certain aquatic diseases. Additionally, the composition and/or combination may improve the feed conversion rate of an aquatic animal. A feed conversion rate, also known as a feed conversion ratio, is a measure of an animal's efficiency in converting feed mass into increased body mass. Animals with low feed conversion rates are considered efficient, as they require less feed to reach a desired weight. Feed conversion rates vary from species-to-species. For example, tilapia typically have a feed conversion ratio of from 1.6 to 1.8, and farm raised salmon typically have a ratio of around 1.2. In some embodiments the feed conversion rate may be enhanced by administering the composition and/or combination by from 0.5% to 20% or more, such as from 1% to 20%, preferably from 2% to 10%, and in certain embodiments, from 3% to 5%.

For some embodiments, such as with aquatic animals, the composition and/or combination can be administered based on body weight, such as grams of the composition and/or combination per pound or kilogram body weight of fish per day, or in milligrams of the composition and/or combination per pound or kilograms of body weight. In a particular example, when administered to fish the composition and/or combination may be provided in a range of from greater than zero to 500 mg per Kg of body weight per day, such as from 10 mg to 350 mg per Kg of body weight per day or from 50 mg to 250 mg per Kg of body weight per day.

Alternatively, the composition and/or combination is administered based on the amount of feed provided to the aquatic animals. In some embodiments, the amount of the composition and/or combination provided to the aquatic animals is from greater than zero to 10,000 mg per Kg of feed or more, such as from 500 mg to 7,500 mg per Kg of feed, or from 1,000 mg to 5,000 mg per Kg of feed.

A person of ordinary skill in the art will appreciate that the amount of the composition and/or combination administered can vary depending upon a number of factors, including the animal species, size of the animal, the age or growth stage of the animal, and type of the feedstuff to which the combination is added. In some embodiments, 100 mg per Kg of body weight per day is administered, and in other embodiments, 200 mg per Kg of body weight per day is administered. In certain embodiments, 1,000 mg, 2,000 mg or 4,000 mg per Kg of feed is administered to the animals.

FIG. 20 provides exemplary ranges for hatchery, nursery and grow-out stages, based on an administration amount of 100 mg of the composition and/or combination per Kg of body weight per day. FIG. 20 illustrates that hatchery stage fish being fed at a feeding rate of 10% of body weight per day and being administered 100 mg of the composition and/or combination per Kg of body weight per day, the dose of the composition and/or combination is 1,000 mg per Kg of feed. This increases to 2,000 mg per Kg of feed for fish at the nursery stage, and up to 4,000 mg per Kg of feed for fish at the grow-out stage. FIG. 20 also provides exemplary feed sizes, of from greater than zero to 1 mm and from 1 mm to 2 mm for the hatchery stage, from 2 mm to 3 mm for the nursery stage, and 3 mm or greater for the grow-out stage. The feed size may vary depending on the species of aquatic animal as well as on the growth stage of the animal. Suitable feed sizes for particular aquatic animals at different growth stages are known to persons of ordinary skill in the art.

In particular disclosed embodiments, the composition and/or combination may be administered to aquatic animals using a carrier and/or coating agent. The carrier and/or coating agent may be any carrier and/or coating agent known to a person of ordinary skill in the art as being suitable for combining with a feed composition and/or combination. In other particular disclosed embodiments, the composition and/or combination may be administered to the aquatic animals using a dispersant or coating agent allowing the composition and or combination to be coupled to the animal feedstuffs in an aquatic environment. In some embodiments, no carrier or coating agent is necessary, and/or the composition and/or combination may be administered as a primary feedstuff.

The animal may be an aquatic animal, including but not limited to a fish, crustacean, and mollusk. In some embodiments, the aquatic animal is a fish or a mollusk. In other embodiments, the aquatic animal is not a crustacean. Aquatic animals may be raised for consumption, ornamental uses, or for other reasons.

The fish may be any fish, with exemplary particular species including tilapia, such as Nile tilapia, blue tilapia, Mozambique tilapia, tilapiine cichlids, or hybrids thereof; sea bream, such as sheepshead, scup, yellowfin bream, gilt-head bream, Saucereye porgies, red sea bream, or hybrids thereof; carp, such as common carp, Asian carp, Indian carp, black carp, grass carp, silver carp, bighead carp, or hybrids thereof; salmon, such as pink salmon, chum salmon, sockeye salmon, coho salmon, Atlantic salmon, chinook salmon, masu salmon or hybrids thereof; trout, such as rainbow trout, Adriatic trout, Bonneville cutthroat trout, brook trout, steelhead trout or hybrids thereof; cod, such as Atlantic northeast cod, Atlantic northwest cod, Pacific cod, or hybrids thereof; halibut, such as Pacific halibut, Atlantic halibut, or hybrids thereof; snapper, such as red snapper, bluefish or hybrids thereof; herring, such as Atlantic herring or Pacific herring; catfish, such as channel catfish, walking catfish, shark catfish, Corydoras, basa, banjo catfish, talking catfish, long-whiskered catfish, armoured suckermouth catfish, blue catfish, or hybrids thereof; flounder, such as gulf flounder, southern flounder, summer flounder, winter flounder, European flounder, olive flounder, or hybrids thereof; hake, such as European hake, Argentine hake, Southern hake, offshore hake, benguela hake, shallow-water hake, deep-water hake, gayi hake, silver hake, North Pacific hake, Panama hake, Senegalese hake, or hybrids thereof; smelt; anchovy, such as European anchovy, Argentine anchoita, Californian anchovy, Japanese anchovy, Peruvian anchovy, Southern African anchovy, or hybrids thereof; lingcod; moi; perch, such as yellow perch, balkhash perch, European perch, or hybrids thereof; orange roughy; bass, such as European sea bass, striped bass, black sea bass, Chilean sea bass, spotted bass, largemouth sea bass, Asian sea bass, barramundi, or hybrids thereof; tuna, such as yellowfin tuna, Atlantic bluefin tuna, pacific bluefin tuna, albacore tuna, or hybrids thereof; mahi; mackerel, such as Atlantic mackerel, Short mackerel, Blue mackerel, chub mackerel, king mackerel, Atlantic Spanish mackerel, Korean mackerel, or hybrids thereof; eel, such as American eel, European eel, Japanese eel, short-fin eel, conga eel, or hybrids thereof; barracuda, such as great barracuda, Pacific barracuda, Yellowstripe barracuda, Australian barracuda, European barracuda, or hybrids thereof; marlin, such as Atlantic blue marlin, black marlin, or hybrids thereof; mullet, such as red mullet, grey mulletor hybrids thereof; Atlantic ocean perch; Nile perch; Arctic char; haddock; hoki; Alaskan pollock; turbot; freshwater drum; walleye; skate; sturgeon, such as beluga, Kaluga, starlet, or hybrids thereof; Dover sole or Microstomus pacificus; common sole; wolfish; sablefish; American shad; John Dory; grouper; monkfish; pompano; lake whitefish; tilefish; wahoo; cusk; bowfin; kingklip; opah; mako shark; swordfish; cobia; croaker. In certain embodiments, the term ‘fish’ does not include salmon or trout. In other embodiments, the fish is selected from tilapia, sea bream, carp, cod, halibut, snapper, herring, catfish, flounder, hake, smelt, anchovy, lingcod, moi, perch, orange roughy, bass, tuna, mahi, mackerel, eel, barracuda, marlin, Atlantic ocean perch, Nile perch, Arctic char, haddock, hoki, Alaskan Pollock, turbot, freshwater drum, walleye, skate, sturgeon, Dover sole, common sole, wolfish, sablefish, American shad, John Dory, grouper, monkfish, pompano, lake whitefish, tilefish, wahoo, cusk, bowfin, kingklip, opah, mako shark, swordfish, cobia, croaker, or hybrids thereof.

The composition and/or combination may be provided to any crustacean, including, but not limited to, shrimp, such as Chinese white shrimp, pink shrimp, black tiger shrimp, freshwater shrimp, gulf shrimp, Pacific white shrimp, whiteleg shrimp, giant tiger shrimp, rock shrimp, Akiama paste shrimp, Southern rough shrimp, fleshy prawn, banana prawn, Northern prawn, or hybrids thereof; crab, such as blue crab, peekytoe crab, spanner crab, Jonah crab, snow crab, king crab, stone crab, Dungeness crab, soft-shell crab, Cromer crab, or hybrids thereof; lobster, such as American lobster, spiny lobster, squat lobster, or hybrids thereof; crayfish; krill; copepods; barnacles, such as goose barnacle, picoroco barnacle, or hybrids thereof. In other embodiments, the crustacean is not a shrimp, and/or is selected from crab, lobster, crayfish, krill, copepods, barnacles, or hybrids thereof.

The mollusk may be selected from squid, such as common squid, Patagonian squid, longfin inshore squid, neon flying squid, Argentine shortfin squid, Humboldt squid, Japanese flying squid, Wellington squid, or hybrids thereof; octopus, such as the common octopus; clams, such as hard clam, soft-shell clam, ocean quahog, surf clam, Asari, Hamaguri, Vongola, Cozza, Tellina, or hybrids thereof; oysters, such as Pacific oyster, rock oyster, European flat oyster, Portuguese oyster, or hybrids thereof; mussel, such as blue mussel, freshwater mussel, green-lipped mussel, Asian green mussel, Mediterranean mussel, Baltic mussel, or hybrids thereof; abalone; conchs; rock snails; whelks; cockles; or combinations thereof.

In some embodiments, the animal can be an aquatic animal susceptible to an environmental malady, such as an acute toxicity of ammonia due to the surrounding environment, or heat stress caused by, for example, an elevated or reduced water temperature Ammonia toxicity may occur when an aquatic animal is exposed to an environment with ammonia concentrations of greater than about 2.0 mg/L. In some embodiments the composition and/or combination is administered prior to the animal experiencing ammonia toxicity, and/or while the animal is experiencing ammonia toxicity. In other embodiments, the method can be used to ameliorate signs or symptoms of disease in an animal that is suffering or afflicted with a disease. Exemplary embodiments can comprise administering the combination to an animal to help ameliorate signs or symptoms of both infectious and non-infectious diseases or conditions.

Examples may include the following:
Infectious disease such as Bacteria, Viruses, Fungal agents or toxic Algae;
Environmental disease such as ammonia toxicity, nitrite toxicity, nitrate toxicity, hypoxia, increased levels of suspended solids, changes in salinity levels, hypothermia, hyperthermia or changes in pH levels;
Nutritional disease such as Vitamin deficiencies, mycotoxins or rancid feed; and
Genetic disease such as anatomical disorders, lordiosis or aplasia of fins.

Stress is a condition in which an aquatic species is unable to maintain a normal physiologic state because of various factors adversely affecting its well-being. Some of the more common stress factors induced in aquaculture include:

Chemical stressors, for example, poor water quality such as low dissolved oxygen or improper pH; pollution such as intentional pollution, chemical treatments, accidental pollution, insect spray, or spills; diet composition, such as the type of protein or amino acids; and nitrogenous and other metabolic wastes, such as accumulation of ammonia, nitrate or nitrite;

Biological stressors, for example, population density such as overcrowding; other species of fish resulting in issues of aggression, territoriality and/or lateral swimming space requirements; micro-organisms, such as pathogenic and non-pathogenic organisms; and micro-organisms, such as internal and external parasites;

Physical stressors, for example, temperature, such as hypothermia and hyperthermia—this is one of the most important influences on the immune system of fish; light; sounds; and dissolved gases; and

Procedural stressors, for example, handling; shipping; and disease treatments.

### V. Examples

### Example 1

In this example, the effect of a combination embodiment on animal health is determined. Female Rats (ca. 200 g) are assigned to 14 treatments with six animals per treatment and fed the following diets for a period of 28 days. During this time, rats receive daily feeding and water, and are maintained on a 12 hr:12 hr light:dark cycle at 20° C. The following compositions, components, and/or combinations thereof, are evaluated:

A—Control diet (Harlan Teklad 8604, Harlan Laboratories)

B—Diet containing composition of mineral clay, silica, β-glucans, and/or mannans

C—Diet containing yucca

D—Diet containing quillaja

E—Diet containing a direct-fed microbial

F—Diet containing β-glucans

G—Diet containing a vitamin D species

H—Diet containing a plant extract

I—Diet that is a mixture of B+C

J—Diet that is a mixture of B+D

K—Diet that is a mixture of B+E

L—Diet that is a mixture of B+F

M—Diet that is a mixture of B+G

N—Diet that is a mixture of B+H

On Day 28, animals are anesthetized with a mixture of ketamine and xylazine and a sample of whole blood (preserved with acid citrate dextrose [ACD]) is taken via cardiac puncture. Rats are then euthanized via cervical dislocation. Whole blood is divided into two fractions: a fraction preserved in “Tempus tubes” for later RNA purification; and a second fraction stored at 4° C. for several hours prior to centrifugation and recovery of the plasma fraction. Purified RNA is stored at −80° C. until it is used for quantitative PCR and plasma will be stored at the same temperature until quantification of suitable biomarkers is possible.

The effects of the 13 additive combinations (items B through N, above) are determined for their ability to affect biomarkers, such as any one or more of the biomarkers disclosed herein, relating to the following biological functions:

a. Surfactant;

b. Emulsification;

c. Anti-oxidant;

d. Anti-inflammatory;

e. Anti-microbial;

f. Anti-hypertensive;

g Immune modulation;

h. Metabolic regulation;

i. Nutrient utilization and transport;

j. Endocrine and neuroendocrine regulation; and/or

k. Longevity

Data is analyzed by analysis of variance (ANOVA) and the effects of individual treatments on outcomes “a” through “k” is assessed with an appropriate multiple range test. Statistically significant interactions are determined wherein individual ingredients (e.g., items B through H, above) have little or no effect on an outcome but have large (potentially synergistic) effects when fed in combination with one another (e.g., Treatments “I” through “N”). The present example can be modified to determine the effects of other combinations (e.g., a combination of B-H and/or a combination of B and any two or more of C-H).

### Example 2

In this example, an embodiment of the combinations disclosed herein (e.g., extracted grape pomace, alone or in combination with a composition comprising β-glucan (e.g., β-1,3 (4)glucan), silica, mineral clay, mannans, and β-1,3 (4)-endoglucanohydrolase, referred to as “Composition A”) was used to assess effects of the combination on biomarkers of immune function on biomarkers of immune function. One embodiment of the present example was a short-term study lasting 6 days and another embodiment was a longer-term study lasting 28 days.

In both embodiments, four groups of male CD rats (8 rats/treatment, 175-200 g) were assigned to four treatments as follows:

1. Control-fed

2. Composition A-fed

3. Grape pomace extract-fed

4. Combination of “2” and “3”

Composition A was provided to Groups 2 and 4 at 0.5% w/w of the diet (Harlan Teklad 8604, Harlan Laboratories). Grape pomace extract was provided to Groups 3 and 4 to provide a daily dose of 6.25 mg of total polyphenol/rat/day. Animals were maintained on these treatments for 6 or 28 days after which they were anesthetized, and blood samples were taken via cardiac puncture.

Neutrophils were purified from blood samples via Percoll gradient centrifugation after which zymosan-stimulated reactive oxygen species (ROS) generation was assessed in a live cell assay. RNA was extracted from neutrophils as well, and concentrations of three mRNAs were assessed using quantitative reverse-transcriptase polymerase chain reaction (qRT-PCR). Quantification included L-selectin, interleukin-8-receptor (IL8R) and β-actin mRNAs. L-selectin and IL8R mRNAs were expressed as a proportion of a house-keeping gene (β-actin) according to the method of Livak and Schmittgen (Methods 25, 402-408 (2001)).

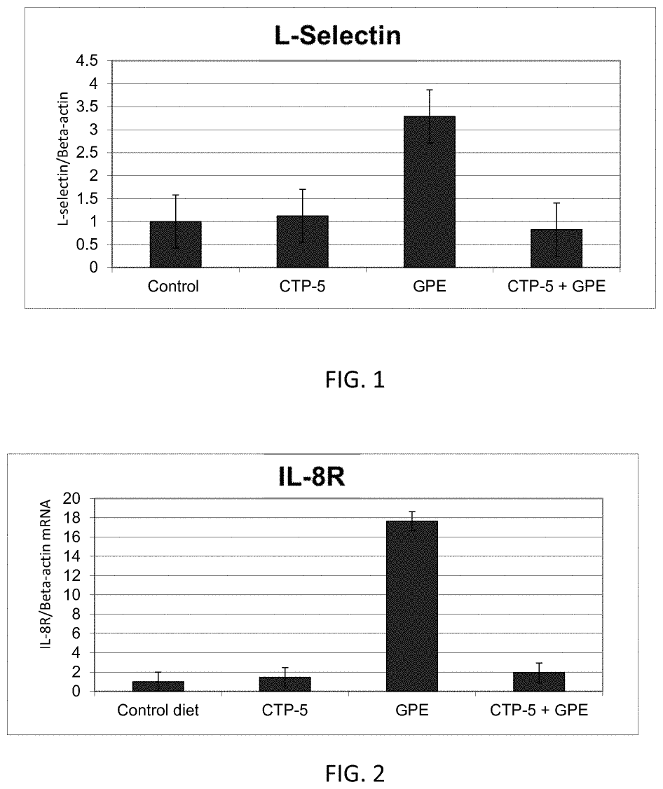

Effects of short-term (6-day) exposure of rats to these four treatments on neutrophil L-selectin and IL8R mRNAs are shown in FIGS. 1 and 2. Feeding Composition A (CTP-5) alone caused small increases in concentrations of both L-selectin and IL8R mRNAs. However, surprisingly, feeding the grape pomace extract (GPE) alone caused massive increases in the expression of both of these biomarkers (FIGS. 1 and 2). These effects of the extract were very unexpected and judged to be undesirable given their magnitude. However, the addition of Composition A to the grape pomace extract product attenuated the stimulatory effects of grape pomace extract on neutrophil biomarker gene expression. These were unexpected interactions.

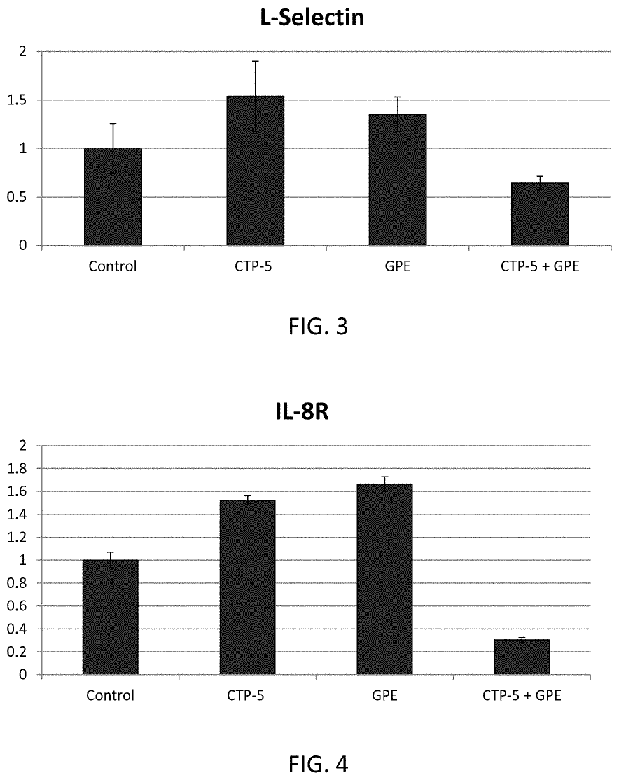

Similar unexpected interactions were also noted in the long-term (28-day) study. In this embodiment, both the Composition A and grape pomace extracts individually augmented expression of both L-selectin and IL8R mRNAs in isolated rat neutrophils. However, the combination of the grape pomace extract and Composition A caused an interaction to occur with consequent reduction in expression of both L-selectin and IL8R mRNAs in isolated neutrophils (FIGS. 3 and 4).

As shown in FIGS. 1-4, Composition A (CTP-5), when fed alone, increased expression of two inflammatory markers: L-selectin and IL8R. Surprisingly, the grape extract product, even though considered to be an anti-inflammatory product, also exerted inflammatory properties in the neutrophils. These data were completely unexpected because antioxidant polyphenols are regarded to function as anti-inflammatory agents. In fact, dozens of studies have provided evidence of this. However, the results shown in FIGS. 1-4 clearly demonstrate that the grape polyphenolic extract, when fed alone, exerted pro-inflammatory properties in the rat model. When Composition A and the grape polyphenolic product were combined, however, a surprising interaction occurred. Specifically, the combination product exerted exactly the opposite effect on neutrophil inflammatory biomarkers than the individual components exerted when provided alone. The combined product reduced expression of two key inflammatory biomarkers in neutrophils.

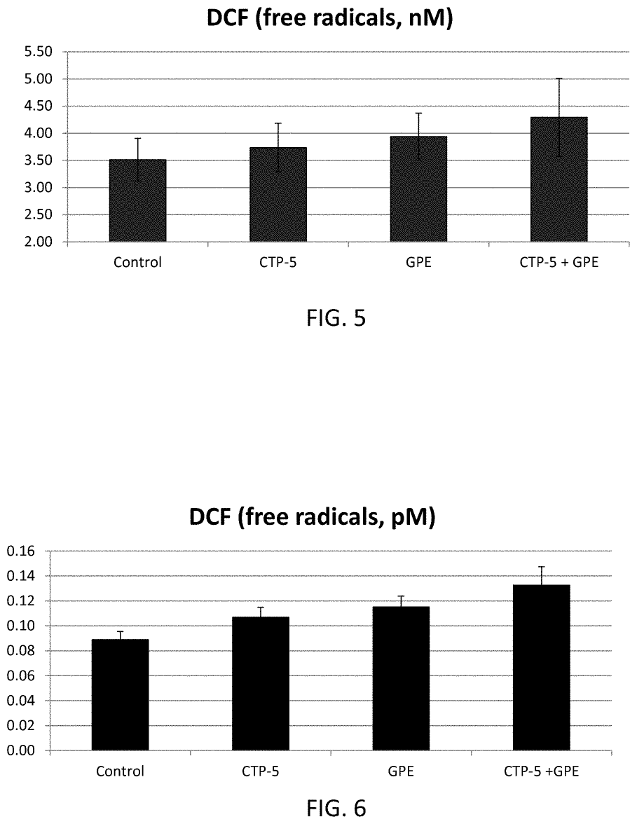

The effects of the four treatments on the neutrophils' ability to generate reactive oxygen species (ROS) in response to addition of zymosan (a pathogen mimetic) following exposure of animals to the treatments for 6 and 28 days was determined. As shown in FIG. 5, exposure of animals to Composition A (CTP-5) for 6 days caused a small (5%) increase in ROS generation ability of neutrophils. Similarly, exposure of animals to the grape pomace extract (GPE) caused an 11% increase in zymosan-stimulated ROS activity. However, neutrophils recovered from animals fed the combination of Composition A and the grape pomace extract demonstrated a synergistic response of their neutrophils to a zymosan challenge. Neutrophils isolated from these rats demonstrated a 23% increase in zymosan-stimulated ROS generation; a wholly unanticipated synergistic interaction.

Similar responses were noted in zymosan-stimulated ROS activity in neutrophils recovered from animals fed the experimental treatments for 28 days (FIG. 6). In long-term fed animals, both Composition A and the grape pomace extract (GPE) enhanced ability of neutrophils to produce ROS in response to a zymosan challenge. However, the combination of Composition A and grape pomace extract in the diet produced a much larger response in ability of neutrophils to produce ROS.

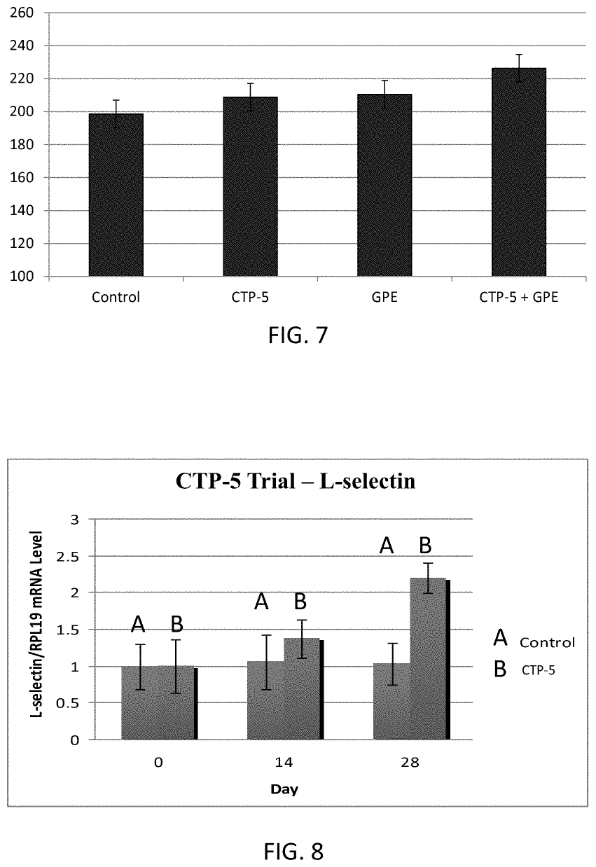

In addition to the unusual and unanticipated effects of the combination product on neutrophil gene expression and ROS generation, interactions between these two feed additives were noted in other assays as well. For example, when animals were fed the treatments for 6 days, serum C-reactive protein (CRP) expression responded in an unanticipated manner Whereas both Composition A (CTP-5) and the grape extract (GPE) exerted small elevations in serum CRP concentrations, the combination of Composition A and grape extract exerted synergistic effects on serum CRP concentrations (FIG. 7). Surprisingly, none of the additives, alone or in combination, had any effect on serum CRP concentrations in the longer-term study (data not shown).

C-reactive protein is an acute-phase protein, which is synthesized in the liver and plays an important role in initiating the complement cascade. Effects of Composition A (CTP-5) in combination with grape extract on CRP expression imply that the combination product would synergistically enhance complement function. These synergistic responses mimic those detected with the combination product's effects on zymosan-mediated ROS generation. Hence, the combination product appears to surprisingly attenuate some aspects of the inflammatory response (e.g., neutrophil recruitment potential) while, at the same time synergistically enhancing other elements of the immunological response (e.g., ROS generation and hepatic formation of CRP/complement activation).

### Example 3

In this example, the effects of Composition A (discussed above) on immune biomarkers in dogs, particularly Beagle dogs, was determined. Specific markers that were analyzed included neutrophil L-selectin and IL-8R mRNAs and plasma Interleukin 6 (IL-6), Interleukin 4 (IL-4), C-reactive protein (CRP) and interferon-γ (INF-γ).

Animals:

Twenty eight, 18-24 month old intact female Beagle dogs were randomly divided into two groups: Group A (n=14; treated) and Group B (n=14; control). The control diet was Nutro® Natural Choice® Lamb Meal and Rice natural dry dog food for adult dogs. The treated diet was Nutro® Natural Choice® Lamb Meal and Rice natural dry dog food for adult dogs supplemented with Composition A at a rate of 0.5% (w/w) of diet fed/day. The dogs were housed in two separate rooms with seven animals from each group kept in each room. All dogs were individually housed and fed on raised decks. A daily log was kept for each dog detailing the amount of food consumed, consistency of bowel movements (fecal consistency score 1-7) and the incidence of other health related symptoms displayed throughout the study. Dogs were weighed and assigned a body condition score (BCS) on Day 0 and Day 28.

Prior to the initiation of the trial described in this example, dogs were being fed Teklad canine diet #8653. Therefore, the dogs were given a six-day acclimation period to the control diet. During this time, the dogs' initial diet was combined with the control diet, gradually decreasing the amount of the dogs' initial diet and increasing the amount of the control diet each day so that by the end of the six day acclimation period all dogs were consuming entirely the control diet.

Sample Collection and Assessment of L-Selectin and IL-8R mRNAs and Plasma IL-6, IL-4, CRP and IFN-γ Concentrations:

Blood samples were collected from the jugular vein from all animals on Day 0 (prior to CTP-5 supplementation), Day 14 and Day 28 of feeding.

One 10 ml sodium heparin tube was collected from each animal at each of the three time points. Neutrophils were immediately isolated from the sodium heparin tube using Percoll gradient centrifugation. RNA from neutrophils was isolated using the Trizol method and stored at −80° C. until further analysis. Concentrations of L-selectin, IL-8R and RPL-19 mRNAs were assessed using quantitative RT-PCR and primers specific for canine sequences. One ml of plasma was frozen at −80° C. for future cytokine analysis. In this sample, concentrations of IL-6, IL-4, C-reactive protein (CRP) and interferon-γ (IFN-γ) were assessed by ELISA. Differences between treatments were generally assessed using a Student's t-test. For assessment of IL-6 and CRP, time-zero values of plasma cytokines were also used as covariates in ANOVA to account for the initial differences in concentrations of IL-6 and CRP noted in the two experimental groups.

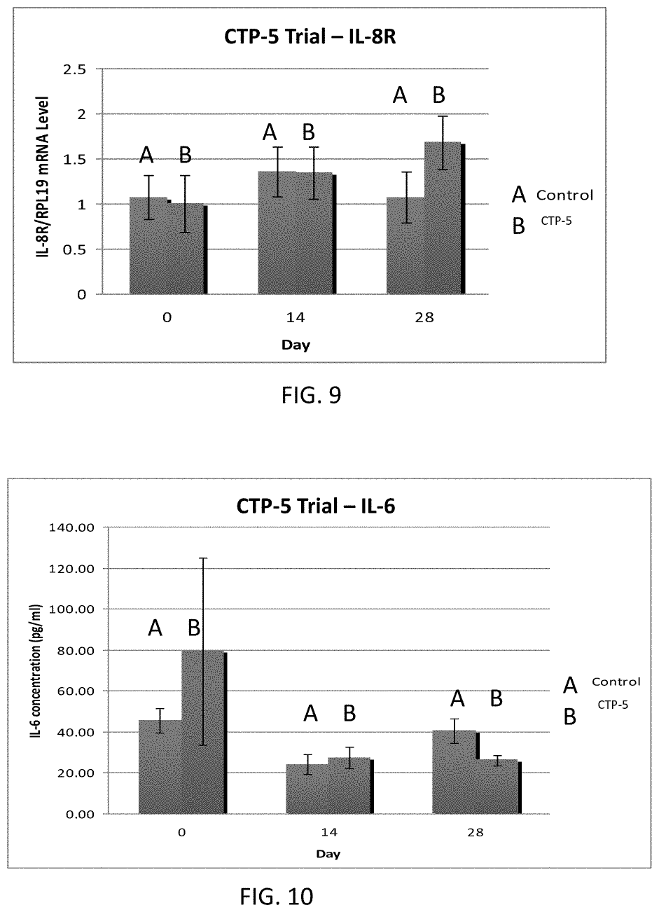

Effects of the two diets on L-selectin and IL-8R mRNAs on Days 0, 14 and 28 are shown in FIGS. 8 and 9, respectively. Feeding Composition A for 14 days had no effect (P>0.05) on neutrophil L-selectin or IL-8R mRNAs; however, on Day 28, animals fed Composition A exhibited a 2.2-fold increase (P=0.004) in neutrophil L-selectin mRNA and a 1.7-fold increase (P=0.029) in IL-8R mRNA.

Effects of the treatments on plasma IL-6 are shown in FIG. 10. No differences between treatments (P>0.05) were detected on Day 14 and Day 28. Concentrations of IL-6 at the beginning of the study, for unknown reasons were highly variable.

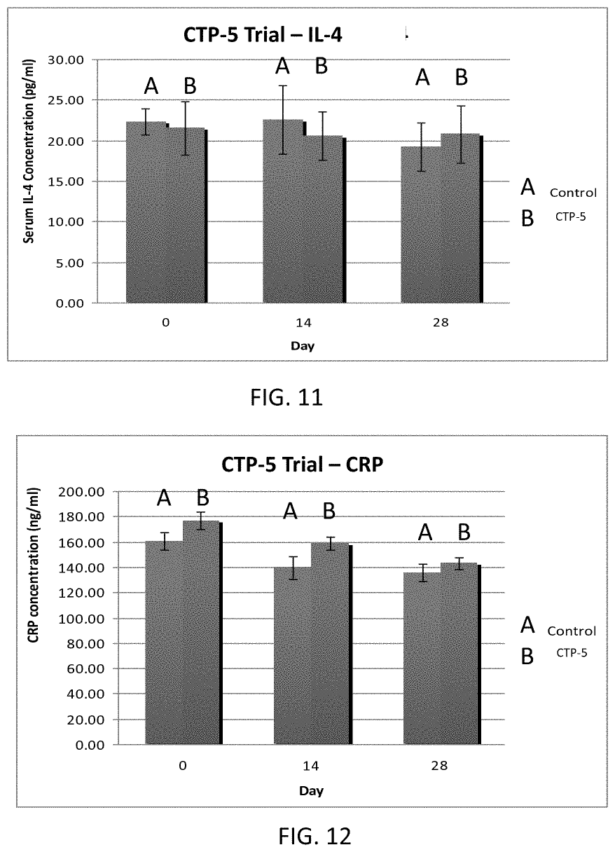

FIG. 11 displays the effects of Composition A on plasma IL-4 concentrations. There were no differences (P>0.05) in IL-4 concentrations between treatments at any of the three time points.

Effects of the treatments on plasma CRP concentrations are shown in FIG. 12. On Days 0, 14 and 28, dogs fed the Composition A exhibited numerically higher CRP concentrations; however, these results were not significant (P>0.05).

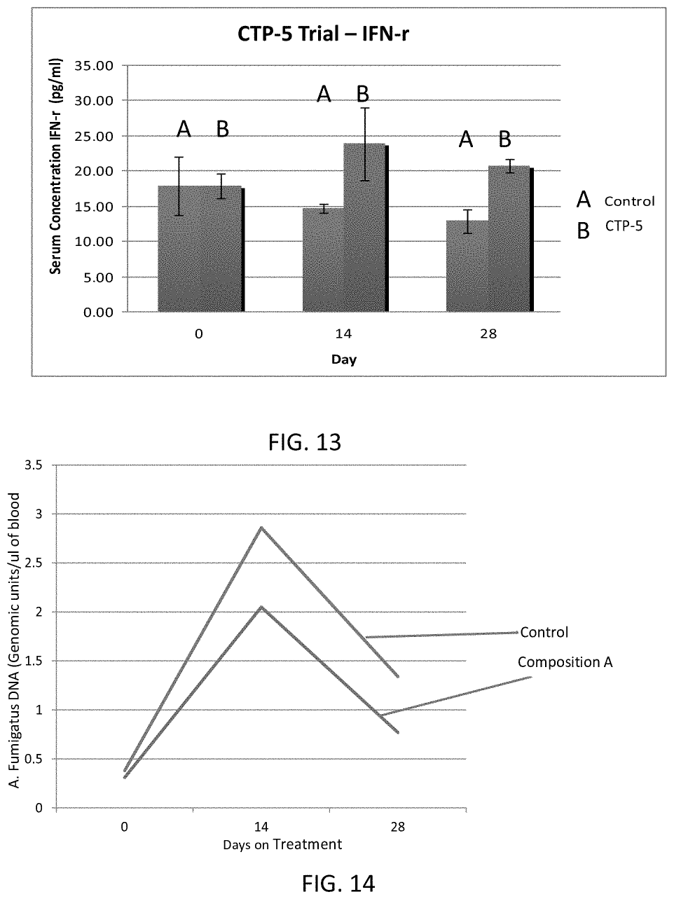

Effects of the treatments on plasma IFN-γ concentrations are shown in FIG. 13. IFN-γ was equal in the control- and Composition A-fed animals at the beginning of the study (Day 0). However, on both Days 14 and 28, Composition A had increased plasma IFN-γ significantly (P<0.05).

Weight and body condition scores did not differ between the groups on Day 0 or Day 28. There were no concerns with palatability of Composition A and no observations of diet-induced adverse clinical signs, such as vomiting, diarrhea, or lethargy in any of the treated animals.

Feeding Composition A increased expression of three biomarkers of innate immunity: neutrophil L-selectin and IL-8R mRNAs and serum IFN-γ. Composition A had no effect on three other biomarkers: IL-6, IL-4 and C-reactive protein. The effects of Composition A on neutrophil L-selectin and IL-8R required more than two weeks to develop. An increase in these two biomarkers implies that the neutrophil would be more sensitive and more “recruitable” to an infection site. Specifically, IL-8 is secreted by macrophages in the vicinity of an infection and serves to recruit leukocytes (including neutrophils). Leukocytes travel via a concentration gradient of IL-8 via detection provided by the IL-8 receptor. Increased expression of IL-8R implies a heightened surveillance function within the neutrophil. L-selectin is expressed in leukocytes (including neutrophils) as an adhesion molecule. L-selectin enables the neutrophil to adhere to the endothelial lining of the capillary and to ultimately escape the vasculature via extravasion. Immunosuppression clearly down-regulates L-selectin expression in neutrophils and represents a means by which stress increases the likelihood of an infection. Increased L-selectin expression in this example similarly implies an enhanced neutrophil surveillance function.

IFN-γ is a cytokine secreted by Th1 cells, cytotoxic T cells and natural killer cells (NK cells). It has antiviral, immunoregulatory and anti-tumor properties. More specifically, IFN-γ promotes NK cell activity, increases antigen presentation and lysosome activity by macrophages, promotes Th1 differentiation, and supports adhesion and binding for leukocyte migration. In this example, IFN-γ concentrations were elevated within 2 weeks of feeding the product. Heightened IFN-γ, as with L-selectin and IL-8R, imply enhances immune surveillance.

IL-6 is released by T-cells, macrophages and endothelial cells in response to infection, neoplasia, trauma and burns. It is an important mediator of fever and of the acute phase response. IL-6 has major effects on hematopoiesis and thrombopoiesis and appears to be a growth factor of malignant cells. The over expression of IL-6 can lead to autoimmune disease and many lymphoid malignancies. The observation that Composition A fed dogs exhibited higher plasma levels of IL-6 on Day 0 (only) suggests that these animals were possibly suffering from a transient sub-clinical infection or trauma as all animals appeared and behaved in a healthy manner on this date. By Day 14 and Day 28, the Composition A fed dogs plasma IL-6 levels were not different from levels found in control fed dogs. In this example, there were also no differences in plasma IL-4 levels between Composition A fed and control fed dogs on Day 0, Day 14 or Day 28. IL-4 is a cytokine that induces differentiation of naïve helper T cells to Th2 cells. It is involved in the regulation of B-cell development, IgE synthesis, and the allergic response. Overproduction of IL-4 is associated with allergies. The fact that Composition A did not increase plasma levels of IL-6 or IL-4 in this study is encouraging and supportive of the conclusion that this product does not over-stimulate the immune system of clinically normal animals.

CRP is an acute phase protein produced by the liver. The physiologic role of CRP is to bind phosphocholine expressed on the surface of dead or dying cells in efforts to activate the compliment cascade. Levels of CRP rise dramatically in response to a wide range of acute or chronic inflammatory conditions such as bacterial, viral, and fungal infections, malignancies and tissue injury. IL-6 can induce the synthesis of CRP by the liver. In this example, plasma levels of CRP between Composition A fed and control fed dogs were measured.

### Example 4

In this example, neutrophil-mediated killing of Streptococcus equi, neutrophil IL-8R mRNA and plasma levels of Aspergillus fumigatus DNA in horses fed Composition A was measured.

Eighteen horses were assigned to one of two groups: 1) control-fed (n=9) and 2) Composition A fed (n=9). Group 1 consisted of 6 geldings and 3 mares which ranged in age from 1 to 16 years. Group 2 consisted of 6 geldings and 3 mares which ranged in age from 2 to 17 years. Horses in Group 1 were fed 4 lb/h/d of a complete feed pellet (18% crude protein, 2% crude fat and 6% crude fiber on an as-fed basis) and timothy hay. Horses in Group 2 received the same diet but with 49 g/h/d of Composition A included in the pellet. Diets were fed for a total of 28 days. Horses were housed in stalls and were ridden daily. Breeds of horses included in the study were Quarter horse, Thoroughbred and Appaloosa, and their weights ranged from 800 lbs. to 1400 lbs. Blood samples were collected from all horses prior to group assignment (Day 0) and on Day 14 and Day 28 of the study. Neutrophils were isolated using Percoll gradient centrifugation and RNA was isolated using Trizol reagent. The amount of IL-8R mRNA was measured with quantitative RT-PCR and based on expression of the reference gene RPL-19. The ability of neutrophils to phagocytose S. equi was assessed on Day 14 and Day 28 and measured by genomic units (GU). Neutrophil killing ability was assessed at two ratios of neutrophils:S. equi (1:30 and 1:60). Plasma was analyzed for A. fumigatus DNA using quantitative RT-PCR. Data were analyzed using PROC GLM (SAS, Statistical Analysis Systems) with significance evaluated at the P<0.05 probability level.

The concentrations of A. fumigatus DNA found in the plasma of horses are shown in FIG. 14. Minimal amounts of A. fumigatus DNA were detected at the beginning of the study (Day 0); however, concentrations were elevated on both Day 14 and Day 28. Concentrations of A. fumigatus DNA in control and Composition A fed horses on Day 14 were 2.86 and 2.05 GU×10⁴, respectively. One horse from Group 1 exhibited very high A. fumigatus DNA concentrations (5.38 GU×10⁴/ml) on Day 14 and died prior to collection of the next blood sample. A. fumigatus DNA in the remaining horses on Day 28 were 1.34 and 0.77 GU×10⁴/ml in control and Composition A fed animals, respectively. A. fumigatus DNA GU means were different across time between control and Composition A horses (P=0.03).

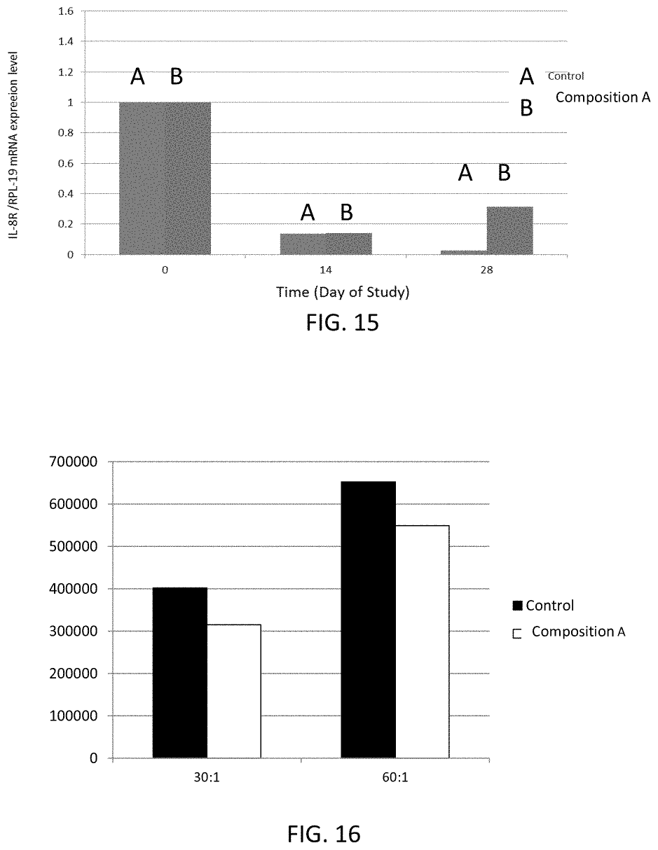

Neutrophil IL-8R mRNA from control and Composition A fed horses is shown in FIG. 15. On Day 14, neutrophil IL-8R mRNA expression was not different between control and Composition A fed horses but was lower in both groups when compared to Day 0 values. On Day 28, neutrophil IL-8R mRNA had declined further in control-fed animals; however, horses fed Composition A had significantly elevated (P=0.01) neutrophil IL-8R mRNA.

Neutrophil-mediated killing of S. equi from control and Composition A fed horses is shown in FIG. 16 [colony forming units (CFU)] and Table 1 [absorbance (550-665 nm)]. Neutrophils obtained from Composition A fed horses killed more S. equi when compared to control fed horses. This effect was significant at both dilutions of 30:1 and 60:1 bacteria:neutrophil [P=0.003 and =0.01 (CFU), and P=0.04 and =0.015 (absorbance), respectively].

TABLE 1
Absorbance (550-665 nm) following neutrophil
phagocytosis of Streptococcus equi

| | |
|---|---|
| 30:1 Ratio Strep | 60:1 Ratio Strep |
| equi:Neutrophil* | equi:Neutrophil** |

| | Day 14 | Day 28 | Day 14 | Day 28 |
|---|---|---|---|---|
| Control | 0.2353 | 0.2735 | 0.3478 | 0.3944 |
| Composition A | 0.2218 | 0.2314 | 0.3163 | 0.3446 |

*Means are different between control and treatment (P = 0.04);
**Means are different between control and treatment (P = 0.015)

In this example, plasma levels of A. fumigatus DNA, neutrophil IL-8R mRNA and neutrophil-mediated killing of S. equi were assessed in 18 horses supplemented with or without Composition A for 28 days. Horses fed Composition A had reduced concentrations of A. fumigatus, increased neutrophil IL-8R mRNA on Day 28 of supplementation and increased neutrophil phagocytic ability compared to controls.

Expression of neutrophil IL-8R mRNA declined in both groups on Day 14 when compared to Day 0 of the study. This decrease might be attributed to cold stress as the weather was unusually cold around Day 14. In addition, there was an accumulation of A. fumigatus DNA in the plasma of horses on Day 14. The source of the A. fumigatus was likely the feed and the presence of this mold may have caused immunosuppression from mycotoxins produced by this fungus. A control fed horse that had the highest concentrations of A. fumigatus DNA on Day 14 was euthanized prior to Day 28 due to signs of colic. A necropsy was performed at the Oregon State University Veterinary Diagnostic Lab and no definitive cause of the abdominal pain was determined. This underscores the possibility that exposure to A. fumigatus may have significant health risks for horses.

Horses commonly endure multiple stressful events throughout their lives, and these stressors can be associated with a reduction in immune function and an increased susceptibility to diseases. In this study, neutrophils obtained from horses fed Composition A exhibited both increased expression of the immune marker IL-8R mRNA and an increased ability to kill a common equine pathogen, S. equi. Without being limited to a particular theory, it is currently believed that supplementing horses with Composition A helps support normal immune function and can result in healthier animals that are better able to tolerate the negative effects of stress.

### Example 5

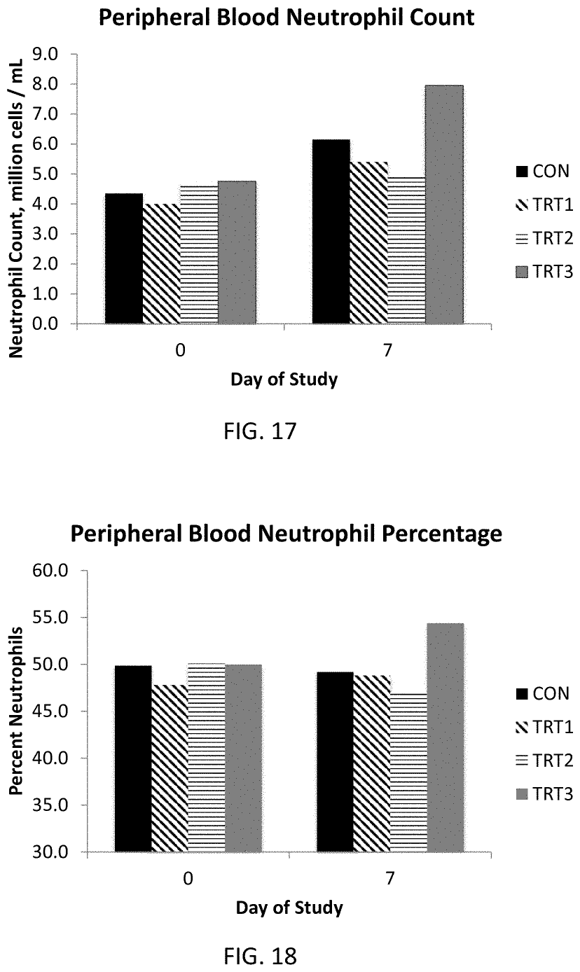

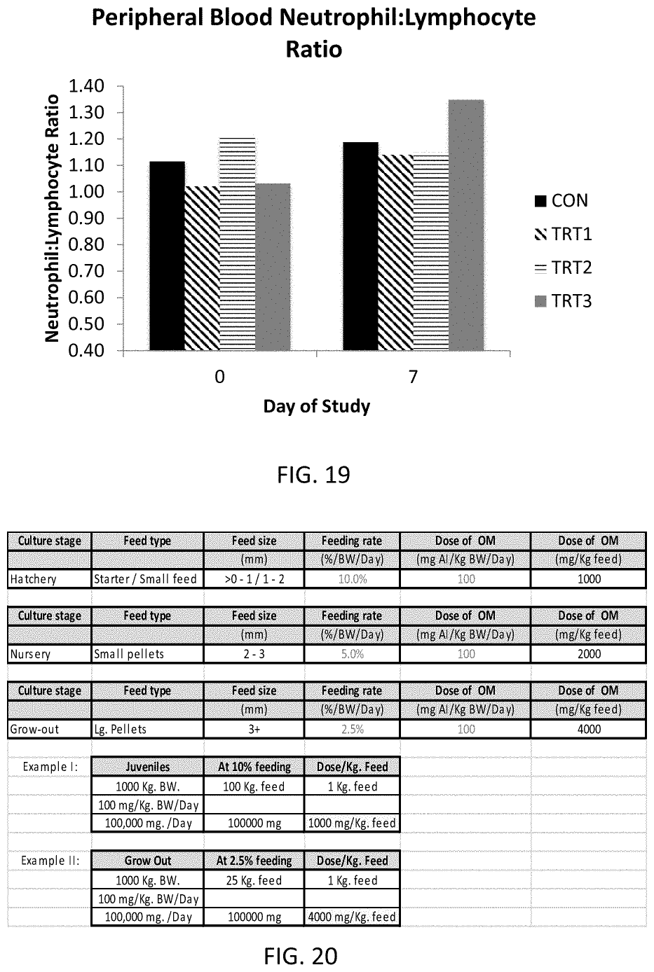

An study was conducted with male dairy calves starting at 1-3 days of age at approximately 30 kg where 45 animals were assigned to one of four treatments: a) a control (CON) treatment, b) a treatment (TRT1) in which a mineral clay and silica and beta-glucan supplement was added to the diet at 0.5% (w/w), c) a direct-fed microbial (DFM; TRT2) and d) a combination of the ingredients listed in “b” and “c” (TRT3). Animals were maintained on diets for a period of seven days. During this period of time growth rate and measurement of immune function of the animals were assessed. At day 0 and day 7, blood was collected from the animals and several assessments of immune function including neutrophil number, neutrophil percentage and peripheral blood neutrophil to lymphocyte ratio were completed. No difference in growth rates were detected in this time period. As can be seen in FIGS. 17-19 results indicated that Treatment “b” exerted a no increase in neutrophil number; neutrophil percentage and peripheral blood neutrophil to lymphocyte ratio neutrophil ROS and Treatment “c” had no effect of these parameters. A strong, synergistic and unexpected effect, however, was observed on neutrophil number, neutrophil percentage and peripheral blood neutrophil to lymphocyte ratio caused by the combination product (Treatment “d”) with these animals exhibiting a 20-40% increase in these parameters (see FIGS. 17-19).

### Example 6

In this example, studies are conducted with female rats weighing approximately 200 g. 32 animals are assigned to one of four treatments: a) a control (CON) treatment, b) a treatment (TRT1) in which a mineral clay and silica and beta-glucan supplement was added to the diet at 0.5% (w/w), c) a yucca and quillaja extract both at 0.0125% (w/w;TRT2) and d) a combination of the ingredients listed in “b” and “c” (TRT3). Rats are maintained on diets for a period of 28 days. During this period of time, growth rate and measurement of inflammation of the animals is assessed. For example, at day 0 and day 28, blood can be collected from the rats and levels of Interleukin 6 (IL-6) can be determined. In some embodiments, a decrease in serum IL-6 can be observed.

### Example 7

In this example, studies will be conducted with female rats weighing approximately 200 g. 32 animals are assigned to one of four treatments: a) a control (CON) treatment, b) a treatment (TRT1) in which a mineral clay and silica and beta-glucan supplement was added to the diet at 0.5% (w/w), c) a yucca and quillaja extract both at 0.0125% (w/w;TRT2) and d) a combination of the ingredients listed in “b” and “c” (TRT3). Rats are maintained on diets for a period of 28 days. During this period of time, growth rate and measurement of inflammation of the animals is assessed. For example, at day 0 and day 28, blood can be collected from the rats and levels of Interleukin-10 (IL-10) can be determined. In some embodiments, a decrease in serum IL-10 can be observed.

### Example 8

In this example, studies are conducted with female rats weighing approximately 200 g. 32 animals are assigned to one of four treatments: a) a control (CON) treatment, b) a treatment (TRT1) in which a mineral clay and silica and beta-glucan supplement was added to the diet at 0.5% (w/w), c) a direct fed microbial (DFM) and d) a combination of the ingredients listed in “b” and “c” (TRT3). Rats are maintained on diets for a period of 28 days. During this period of time, growth rate and measurement of mid-villus brush border surface area of the jejunum of the animals is assessed. For example, at day 0 and day 28, tissue can be collected from the rats and a histological examination of the tissue can be completed to determine whether any increase in brush border surface area is observed in the jejunum.

### Example 9

In this example, the composition and/or combination was administered as a composition comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan. Embodiments of the composition were used as a feed additive for sea bream. Juveniles of Gilthead sea bream (Sparus aurata) were stocked in 12 tanks in the experimental station. Each tank of 1.0 cubic meters was stocked with 55 juvenile sea bream at an average weight of 26 grams. The water source was from a well at a stable temperature of 21° C., with a constant total salinity of 18.0 parts per thousand. The duration of the experiment was 158 days.

The experimental protocol included continuous assessment for the presence of diseases causing organisms. Growth performance parameters of the fish were recorded regularly. The daily/weekly assessment of water quality parameters included ammonia, nitrite, pH, temperature and oxygen.

Feeding rate was based on the recommended commercial feeding chart of Phibro Aqua and adjusted according to the size of the fish and the water temperature. Feeding was performed manually twice a day. The feeding quantity for each tank was adjusted after evaluating the average weight of the fish in each tank every two weeks.

The composition was top-coated on the pellets using 2 wt % of soy oil as a coating agent. The control group was given the same feed coated with 2 wt % soy oil. The feed preparation for the trial included mixing the weighted feed in a mixer for 5 minutes with 2 wt % soy oil, and then additional 5 minutes coating with the composition. The experiment was carryout in replicates of 4 tanks per treatment. In the trial 2 different doses of the composition in the feed were compared: 100 milligrams per kilogram of bodyweight per day; and 200 milligrams per kilogram of bodyweight per day.

The feed used in this trial was made by Raanan Fish Meal and was based on sinking extruded pellets #4932S0 at sizes of 2-4 millimeters; containing 45.0% protein, 12.0% fat, 3.0% carbohydrates, 9% ash and 9.8% moisture.

General health parameters:

1. Survival rates in all the tanks for all the treatments were high (99.1-99.5%).

2. No external or internal parasites were detected in the trial.

3. The general health condition as indicated by the vitality and the response to the feeding was very good for all the treatments for the entire trial.

A significantly higher growth rate of the fish fed with dose (A), using 100 milligrams of the composition per kilogram of bodyweight per day was obtained in this trial. A better growth rate in treatment (A) was observed by day 17. This difference became statistically significant by day 59. Without being bound to a particular theory, the better growth rate may be due to an improved nutrition for the fish and/or improved immunostimulant ingredients in the feed.

As shown in this trial, the response of the fish to the composition and/or combination was significantly better compared to a control group without the composition and/or combination. This conclusion emphasized the efficacy and the advantage of the composition and/or combination as an effective feed additive in aquatic animals such as fish.

Treatment (A) had the lowest significant feed conversion ratio (FCR) value among the 3 treatments. This demonstrated the advantage of the composition and/or combination as an advanced performer, improving the feed intake ability of the fish. This ability to lower the FCR value is a major factor in aquaculture management, because it reduced the feeding cost, which is often the highest cost for fish and shrimp farmers.

The environmental conditions of this example in terms of water temperature, dissolved oxygen levels and water quality were optimal for rearing sea bream. The growth rates of all the 3 groups were according to the expected growth rate of sea bream. The high percentage of survival (99.1-99.5%) in all 3 groups in this study emphasized the optimum conditions during the trial. The lower temperature at the end of the trial affected the optimal growth rate of the fish but still the advantages of the composition were evident.

### Example 10

In this example, the composition and/or combination was administered as a composition comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan. The composition was used as a feed additive for tilapia. Juveniles of hybrid tilapia (Oreochromis niloticus×O. aureus) were stocked in 18 cages in the experimental station. The total volume of the experimental system was 600 cubic meters. Each cage of 1 cubic meter in volume with a 25 millimeter mesh net was stocked with 35 fish at an average weight of 95 grams. The water source was from a well at a stable temperature of 24° C. The duration of the experiment was 149 days.

The experimental protocol included continuous assessment for the presence of disease causing organisms. Growth performance parameters of the fish were recorded regularly. The daily/weekly assessment of water quality parameters included ammonia, nitrite, pH, temperature and dissolved oxygen.

Feeding rate was based on the recommended commercial feeding chart of Phibro Aqua and adjusted according to the size of the fish and the water temperature. Feeding was performed manually twice a day. The feeding quantity for each cage was adjusted after evaluating the average weight of the fish in each cage every two weeks.

The composition was top-coated on the pellets using 2 wt % soy oil as the coating agent. The control group was given the same feed coated with 2 wt % soy oil. The feed for the trial was prepared by mixing the weighted feed in a mixer for 5 minutes with 2 wt % soy oil, and then additional 5 minutes coating with the composition. In this trial 2 different doses of the composition in the feed were compared: 100 milligrams AI per kilogram of bodyweight per day; and 200 milligram AI per kilogram of bodyweight per day.

Replicates of 6 cages were used per treatment, which were divided equally in the rearing system. The feed for this trial was manufactured by Zemach Feed Mill. The feed is based on floating extruded pellets #4662 at sizes of 2-4 millimeters; containing 35.0% protein, 3.5% fat, 14.0% carbohydrates, 8.0% ash and 10.0% moisture.

General health parameters:

1. Survival rate in all the cages for all the treatments were excellent, without mortality.

2. External parasites (Trichodina and Dactylogyrus) were detected at low incidence. The fish were treated with formalin 37% and Bromex solution (50% Naled).

3. Low presence of dignea parasite, Centrocestus, was detected. No treatment was required.

4. The general health condition as indicated by the vitality and the response to the feeding was very good for all treatments for the entire trial.

A significantly higher growth rate of the fish fed with dose (A), using 100 mg of the composition per kilogram of bodyweight per day was obtained in this trial. A better growth rate in treatment (A) was observed by day 16. This difference became statistically significant by day 86. Without being bound to a particular theory, the better growth rate may be due to an improved nutrition for the fish and/or improved immunostimulant ingredients in the feed.

Administering the composition led to a better growth rate and a better feed intake. 100 and 200 milligrams/kilogram bodyweight per day doses were administered. As shown in this trial, the response of the fish to the composition was significantly better compared to the control group without the composition and/or combination. This conclusion emphasized the efficacy and the advantage of the composition and/or combination as an effective feed additive in aquatic animals such as fish.

Treatment (A) had the lowest significant FCR value among the 3 treatments. This demonstrated the advantage of the composition and/or combination as an advanced performer, improving the feed intake ability of the fish. This ability to lower the FCR value is a major factor in aquaculture management. The feeding cost is often the highest cost for fish and shrimp farmers.

The environmental conditions of the experiment in terms of water temperature, dissolved oxygen levels and water quality were optimal for rearing tilapia. The growth rates of all the 3 groups were better when compared to the expected growth rate of tilapia, emphasizing the optimal conditions of the trial. The high percentage of survival (100%) in all 3 groups in this study also emphasized the optimum conditions during the trial. The lower temperature at the end of the trial affected the optimal growth rate of the fish but still the advantages of the composition and/or combination were evident.

### Example 11

In this example, the composition and/or combination was administered as a composition comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan. The composition was used as a feed additive for carp. Juveniles of Common carp (Cyprinus carpio) were stocked in 18 cages in the experimental station. The total volume of the experimental system was 600 cubic meters. Each cage of 1 cubic meter in volume with a 25 millimeter mesh net was stocked with 35 fish at an average weight of 160 grams. The water source was from a well at a stable temperature of 24° C. The duration of the experiment was 83 days.

The experimental protocol included continuous assessment for the presence of diseases causing organisms. Growth performance parameters of the fish were recorded regularly. The daily/weekly assessment of water quality parameters included ammonia, nitrite, pH, temperature and dissolved oxygen.

Feeding rate was based on the recommended commercial feeding chart of Phibro Aqua and adjusted according to the size of the fish and the water temperature. Feeding was performed manually twice a day. The feeding quantity for each cage was adjusted after evaluating the average weight of the fish in each cage every two weeks.

The composition was top-coated on the pellets using 2 wt % soy oil as the coating agent. The control group was given the same feed coated with 2 wt % soy oil. The feed for the trial was prepared by mixing the weighted feed in a mixer for 5 minutes with 2 wt % soy oil, and then additional 5 minutes coating with the composition. In this trial 2 different doses of the composition and/or combination in the feed were compared: 100 mg per Kg of body weight per day; and 200 mg per Kg of body weight per day.

Replicates of 6 cages were used per treatment, which were divided equally in the rearing system. The feed for this trial was manufactured by Zemach Feed Mill. The feed is based on floating extruded pellets #4212 at size of 4 millimeters; containing 30.0% protein, 5.0% fat, 4.5% carbohydrates, 8.0% ash and 10.0% moisture.

General health parameters:

1. Survival rate in all the cages for all the treatments were excellent, without mortality.

2. External parasites (Gyrodectylus and Dactylogyrus) were detected at low incidence. The fish were treated with formalin 37% and Bromex solution (50% Naled).

3. The general health condition as indicated by the vitality and the response to the feeding was very good for all treatments for the entire trial.

A significant higher growth rate of the fish fed with dose (A), using 100 milligrams of the composition per kilogram of bodyweight per day was obtained in this trial. A better growth rate in treatment (A) was observed by day 41. This difference became statistically significant by day 83. Without being bound to a particular theory, the better growth rate may be due to an improved nutrition for the fish and/or improved immunostimulant ingredients in the feed.

Administering the composition led to a better growth rate and a better feed intake. 100 and 200 milligram/kilogram bodyweight per day doses were administered. As shown in this trial, the response of the fish to the composition was significantly better compared to the control group without the composition and/or combination. This conclusion emphasized the efficacy and the advantage of the composition and/or combination as an effective feed additive in animals like fish.

Treatment (A) had the lowest (insignificant) FCR value among the 3 treatments. This demonstrated the advantage of the composition and/or combination as an advanced performer, improving the feed intake ability of the fish. This ability to lower the FCR value is a major factor in aquaculture management. The feeding cost is often the highest cost for fish and shrimp farmers.

The temperatures of the experiment demonstrated a cold water environment (16-21° C.). This range of temperatures is common in carp culture worldwide. These low temperatures affected the optimal growth rate of the fish but still the advantages of the composition and/or combination were evident. The water conditions in terms of dissolved oxygen levels, ammonia, nitrite and pH were optimal for rearing carp. The high percentage of survival (100%) in all 3 groups in this study emphasized the optimum conditions during the trial.

### Example 12

In this example, the composition and/or combination was administered as a composition comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan. The composition was used as an immune modulator for hybrid tilapia Ammonia is a toxic compound that can adversely affect fish health. The nature and degree of toxicity depends on many factors, including the chemical form of ammonia, the pH and temperature of the water, the length of exposure, and the life stage of the exposed fish. In natural surface waters, ammonia occurs in two forms: ionized ammonia, NH₄⁺, and un-ionized ammonia, NH₃. In fish, ammonia is a byproduct of protein metabolism and is primarily excreted across the gill membranes, with a small amount excreted in the urine. Ammonia's toxicity is principally due to the un-ionized form, NH₃. As pH increases, the toxicity of ammonia rises because the relative proportion of unionized ammonia increases. The toxicity of ammonia may cause convulsions, coma and death. Without being bound to a particular theory, elevated NH₄⁺ in the fish body may displace K⁺ and depolarize neurons, causing activation of glutamate receptor, which leads to an influx of excessive Ca²⁺ and subsequent cell death in the central nervous system. In the case of larvae of common carp, acute toxicity of 1.76 parts per million of NH₃ caused 50% mortality in the group after 24 hours. Chronic effects of ammonia were studied in three batches of turbot (Scophthalmus maximus) juveniles (14, 23 and 104 grams) exposed for 4-6 weeks to constant ammonium chloride solutions, Under the environmental conditions used (16.5-17.5° C., pH 7.92-8.03, salinity 34.5 parts per thousand, over 80% oxygen saturation), no mortalities occurred up to 0.4 parts per million unionized ammonia. In adapted small turbot, no major physiological disturbances were observed up to 0.4-0.5 parts per million, while large turbot were more sensitive to ammonia.

The ability to improve the resistance of aquatic species to the toxicity of the ammonia has been investigated. Tiger shrimp (Penaeus monodon), 5-day post larvae, were fed diets supplemented with 0 and 71.5 parts per million astaxanthin for 8 weeks. Shrimp were then subjected to 72 hours exposure of ammonia at 0.02, 0.2, 2 and 20 parts per million. The survival rates of the astaxanthin-fed shrimp were higher than those of the control shrimp under all levels of ammonia except 20 ppm, showing that the shrimp's resistance to ammonia stress had been improved by dietary astaxanthin. Other research has investigated the effects of dietary mannan oligosaccharide (MOS) on growth performance, gut morphology, and NH₃ stress tolerance of Pacific white shrimp Litopenaeus vannamei. After NH₃ stress for 24 hours, survival rates of shrimp fed 2.0, 4.0, 6.0 and 8.0 grams/kilogram MOS-supplemented diets were significantly higher (P<0.05) than that of shrimp fed a control diet.

The purpose of this study was to evaluate the effect of the composition and/or combination on the fish resistance to the stressful condition of toxic ammonia levels in the water.

Hybrid tilapia (Oreochromis niloticus×O. aureus) were stocked in 12 tanks in the experimental station. Each tank of 230 liter in volume was stocked with 10 fish with an average weight of 350 grams per fish. The water source was from a well with a constant water temperature of 22° C. and constant salinity of 1,300 milligrams chloride. The duration of the experiment was 74 days. During the first phase, 6 tanks were fed 100 milligrams of the composition and/or combination per kilogram of bodyweight per day, while the other 6 tanks were fed with commercial feed without supplement. After 30 days of feeding in optimal conditions of water, the water inlet was reduced, allowing the water quality to deteriorate for an additional 30 days. In the third phase of 14 days the water inlet was closed completely and ammonium chloride (NH₄Cl) was added to each tank on a daily basis. This phase was characterized by a continuous mortality of the fish showing clinical symptoms of ammonia toxicity and bacterial infections associated with poor water quality.

The experimental protocol included continuous assessment for the presence of diseases causing organisms. The daily assessment of water quality parameters included ammonia, nitrite, pH, water temperature and dissolved oxygen.

Feeding rate was 1% of bodyweight, based on the recommended commercial feeding chart of Phibro Aqua and was adjusted according to the water temperature and the response of the fish. Feeding was performed manually twice a day. The composition was top-coated on the pellets using 2 wt % soy oil as the coating agent. The control group was given the same commercial feed coated with 2 wt % soy oil, but without the composition. The feed for the trial was prepared by mixing the weighted feed in a mixer for 5 minutes with 2 wt % soy oil, and then additional 5 minutes coating with the composition. The feed for this trial was manufactured by Zemach Feed Mill. The feed is based on floating extruded 4 mm pellets, #4662; containing 35.0% protein, 3.5% fat, 14.0% carbohydrates, 8.0% ash and 10.0% moisture.

General health parameters:

1. At the third phase (14 days) the fish didn't respond to the feed.

2. The moribund and the dead fish that were collected during the trial had typical clinical symptoms of toxicity of ammonia.

The results of this trial showed a significant higher resistant fish fed a diet with the composition and/or combination at a dose of 100 mg/Kg of body weight per day compared to the control without the composition and/or combination. In this trial, the moribund and the dead fish that were collected during the trial had typical clinical symptoms of ammonia toxicity, including convulsions, gill necrosis, coma, and death.

Poor water quality suppresses the immune system of the fish, enabling parasites and bacteria to enter the fish body, causing disease outbreak and consequently mortality. In the experiment, the clean water inlet flow was reduced to cause deterioration of the water quality, which finally resulted in death in the most stressed and frail fish in this trial.

Feeding tilapia with the composition and/or combination at a dose of 100 milligrams/kilogram of body weight per day for 30 days period significantly improved their resistance and survival under poor water conditions such as high levels of ammonia and nitrite.

### Example 13

In this example, the ability of the composition and/or combination to act as an immune modulator on the survival and the overall health status of the Pacific White Shrimp (Litopenaeus vannamei) was determined

2,400 Postlarva-20 days of Litopenaeus vannamei were stocked in 12 tanks in the experimental station. Each tank of 500 liter in volume was stocked with 200 PL-20, at an estimated weight of 0.15 g per postlarvae. The experimental unit included a central collecting tank and a central biofilter. The water source was from a well. Balance marine salt was added to the water to achieve a total salinity of 10 ppt (parts per thousand). The duration of the experiment was 71 days. The average size of the shrimp at the end of the trial was around 10 grams.

In this trial, the composition and/or combination was administered as a composition comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan. 2 different doses of the composition were compared to a control. 4 tanks were fed 100 mg of the composition per Kg of BW per day, 4 tanks were fed 200 mg of the composition per Kg of BW per day, while the other 4 tanks were fed with commercial feed without supplement.

Feeding rate based on the recommended feeding chart of Phibro Aqua for Shrimp. Feed quantity was adjusted according to the water temperature, the response of the shrimp and the estimation of their average weight. Feeding was performed manually twice a day. The composition was top-coated on the pellets using 2% of Soy oil as the coating agent. The control group was given the same feed without supplement coated with 2% Soy oil. The feed for the trial was prepared by mixing the weighted feed in a cement mixer (maximum load of 50 Kg) for 5 minutes with 2% Soy oil, and then additional 5 minutes coating with the supplement.

The experimental protocol included continuous assessment for the presence of diseases causing organisms. The daily assessment of water quality parameters included total salinity, ammonia, nitrite, pH, water temperature and dissolved oxygen.

The results illustrate that a significantly greater percentage of the shrimp that were fed the supplement survived, compared to the control group. The conditions of the trial were excellent for growing shrimp. Shrimp had good body condition and good coloration. No external parasites were detected. The final average weight of the shrimp was around 10 grams, normal for in-door culture. At this stage (nursery), the survival rates of the control groups (60%) are normal.

In a different trial, 2500 shrimp in a pond were administered a composition comprising between 15% and 40% silica, between 50% and 81% mineral clay, between 1.0% and 5.0% β-glucans, between 0.05% and 3.0% β-1,3 (4)-endoglucanohydrolase and between 1% and 8.0% mannan. After 6 months, the shrimp were compared to 2500 control shrimp in a separate pond that were not administered the composition. The composition-fed shrimp has an 86% survival rate, compared to 22% for the control shrimp, and also had a greater yield (kg/pond) than the control shrimp.

In view of the many possible embodiments to which the principles of the disclosed technology may be applied, it should be recognized that the illustrated embodiments are only of the technology and should not be taken as limiting the scope of the present disclosure. Rather, the scope of the present disclosure is defined by the following claims. We therefore claim all that comes within the scope and spirit of these claims.

1-27. (canceled)

28. A composition, comprising:
mineral clay, silica, β-glucan and mannans; and
a direct-fed microbial, yucca, quillaja, a chromium compound, or a combination thereof.

29. The composition of claim 28, comprising mineral clay, silica, β-glucan, mannans, and a direct-fed microbial.

30. The composition of claim 28, wherein the direct-fed microbial is yeast.

31. The composition of claim 28, wherein the direct-fed microbial is a Bacillus species.

32. The composition of claim 31, wherein the direct-fed microbial is Bacillus coagulans.

33. The composition of claim 28, comprising mineral clay, silica, β-glucan, mannans, and a chromium compound.

34. The composition of claim 28, comprising mineral clay, silica, β-glucan, mannans, yucca, and quillaja.

35. The composition of claim 28, wherein further comprises an endoglucanohydrolase.

36. The composition of claim 28, wherein the composition comprises 1-40 wt % silica, 1-25 wt % glucan and mannans, and 30-92 wt % mineral clay.

37. The composition of claim 28, wherein the composition comprises Quillaja Saponaria, Yucca schidigera or a combination thereof.

38. The composition of claim 28, wherein the composition further comprises a vitamin, a plant extract or a combination thereof.

39. The composition of claim 38, wherein the vitamin is a vitamin D species, a niacin supplement, a vitamin B-12 supplement, biotin, d-calcium pantothenate, choline chloride, thiamine mononitrate, pyridoxine hydrochloride, menadione dimethylpyrimidinol, bisulfate riboflavin-5-phosphate, folic acid, or a combination thereof.

40. The composition of claim 28, further comprising an animal food.

41. The composition of claim 28, comprising:
1-40 wt % silica, 0.5-25 wt % glucan and mannans, 40-92 wt % mineral clay from 0.05 wt % to 3 wt % β-1,3 (4)-endoglucanohydrolase, and yeast.

42. A method, comprising administering to an animal a composition according to claim 28.

43. The method of claim 42, wherein the composition comprises mineral clay, silica, β-glucan, mannans, and a direct-fed microbial.

44. The method of claim 42, wherein the composition comprises mineral clay, silica, β-glucan, mannans, and yeast.

45. The method of claim 42, wherein the animal is a feed animal.

46. The method of claim 42, further comprising administering an animal feed as a composition or a combination.

47. The method of claim 42, wherein the animal is a cow, a pig, a sheep, a canine, a feline, a domestic fowl, a fish, a reptile, a crustacean, a mollusk, a rabbit, a goat, a deer, a bison, a buffalo, an alpaca, a horse, a donkey, or a llama.

48. The method of claim 42, wherein administering the composition to the animal ameliorates at least one deleterious symptom or sign in the animal, or delays onset of the at least one deleterious symptom or sign in the animal.
